(19) United States
(12) Patent Application Publication
(10) Pub. No.: US 2010/0113840 A1
(43) Pub. Date: May 6, 2010

(54) Plants with altered carbon allocation

(76) Inventor: Dean Dellepenna, Williamstown, MI (US)

Correspondence Address: MEDLEN & CARROLL, LLP, 101 HOWARD STREET, SUITE 350, SAN FRANCISCO, CA, 94105, US

(21) Appl. No.: 12/310,722

(22) PCT Filed: Sep. 5, 2007
(86) PCT No.: PCT/US2007/019326

§ 371 Date: Dec. 29, 2009

Related U.S. Application Data
(60) Provisional application No. 60/842,262, filed on Sep. 5, 2006.

(51) Int. Cl. C07C 31/08; A01H 5/00; C12N 15/82

(52) U.S. Cl. 568/840; 800/298; 800/306; 800/320; 800/320.1; 435/320.1

(57) ABSTRACT

The present invention relates to compositions and methods for altering carbon allocation in plants. In particular, the present invention provides for the use of plants with altered carbon allocation in generating biofuels by modifying the carbon allocation in plants such that carbon is preferentially allocated into starch and soluble sugars in plant leaves in lieu of typical carbon sinks, thereby allowing the carbon to be more readily fermentable for use as biofuels.

The present application was funded in part by a National Science Foundation Grant. The government may have certain rights in the invention.

### FIELD OF THE INVENTION

The present invention relates to compositions and methods for altering carbon allocation in plants. In particular, the present invention provides for the use of plants with altered carbon allocation in generating biofuels by modifying the carbon allocation in plants such that carbon is preferentially allocated into starch and soluble sugars in plant leaves in lieu of typical carbon sinks, thereby allowing the carbon to be more readily fermentable for use as biofuels.

### BACKGROUND OF THE INVENTION

The early decades of the 21^{st} century will see a shift in the global economy from one based primarily on petroleum to one increasingly reliant on bio-based and renewable resources. This fundamental change will affect many aspects of life on earth, and perhaps none more deeply that agriculture and the automotive/energy sectors. This change is precipitated by not only the decline of fossil fuels, but also by a growing recognition and acceptance by the world peoples of pollution and environmental changes that occur with the combustion of fossil fuels. As the nations of the world become aware of the need to reduce dependence on petroleum based products, other alternatives are being seriously considered. Wind, solar, geothermal and other such resources have been around for decades and continue to be considered as viable energy alternatives. The political landscape surrounding the global fuel economy seems to be shifting, albeit slowly. The newly announced Advanced Energy Initiative of the United States Government appears to be heading toward the increase in development of clean energy alternatives.

One alternative to clean energy production is through product development in the agricultural sectors of the world. Therefore, what are needed are processes, methods, and compositions for research and development surrounding the use of plants and plant related products in generating usable fuel sources.

### SUMMARY OF THE INVENTION

The present invention provides compositions, methods and processes for modifying the form of carbon accumulation by photosynthetic tissues in crops to increase the amount of carbon allocated to starch and soluble sugars (e.g., glucose, sucrose, fructose). The use of photosynthetic plant tissues (e.g., leaves and above ground structures) for use as feedstock for biofuels requires desegregation and degradation of the most abundant form of fixed carbon; that fixed in plant cells walls as cellulose. Plant cell walls typically account for 90-95% of the fixed carbon in plant tissues and, due to their extreme complexity and recalcitrance to digestion, harvesting of carbon in the cell wall is time and cost intensive because of necessary pretreatments prior to fermentation. Starch and soluble sugars are more readily fermentable than plant cell walls, thereby bypassing the time and cost constraints associated with pre-digestion of the cell wall and other necessary pretreatments prior to fermentation.

The present invention provides methods and compositions to block the transport of fixed carbon from leaves and stems, and redirect it into different carbon sinks within the leaf cells (e.g., starch and soluble sugars). The accumulation of total starch and soluble sugars in a model crop system (e.g., Arabidopsis sp.) can reach levels ranging from around 20-50% of the leaf dry weight. Therefore, it is contemplated that plants can be modified to provide a large store of carbon that can be more easily converted to biofuels (e.g., ethanol).

The impact and response of mutant plants exhibiting modified tocopherol synthesis (mutant plants deficient in tocopherols) to various abiotic stress treatments was analyzed. It was found that the transfer of plants from normal growth conditions (20° C.) to non-freezing cold treatments (0-12° C.) caused plants to grow more slowly and accumulate high levels of starch and soluble sugars. It was determined that the increased accumulation was due to a block in phloem parenchyma cells that is required for transport of carbon from leaves (source of fixed carbon) to the sink (roots and other structures requiring carbon). Because transport is inhibited, the mutant plants accumulate their fixed carbon into different sinks (e.g., starch and soluble sugars). Photosynthetic performance of mutant plants as a measure of carbon fixation relative to cold treated wild type plants was not impacted, with fixed carbon accumulating in leaf tissues. Because the specific induction of the response in mutants occurs due to cold treatment, engineering or breeding of analogous mutations in crop plants will allow cold induced (e.g. late season) accumulation of soluble sugars and starch in leaves that are ideal for biofuel feedstocks. An alternative approach to disrupting the tocopherol synthesis pathway in plant tissues is to target the disruption via RNA interference (RNAi) methods (e.g., siRNA) or other similar approaches for reducing the expression of specific plant genes. Such a disruption could be plant wide, or targeted to specific cell types (e.g., vascular parenchyma cells) by using tissue specific promoters. As well, RNAi could equally be an inducible event, whereby the expression of a plant gene is inhibited due to an environmental cue (e.g., temperature), a developmental cue (e.g., late plant growth), or at any point in development in response to small molecule inducers (e.g., tetracycline, dexamethasone). For example, genes and nucleic acid sequences related to tocopherol synthesis as described in U.S. Pat. Nos. 7,067,647, 6,872,815, 6,841,717, 6,787,683, 6,541,259 and published US patent applications 2005/0120406 and 2004/0018602 (all incorporated herein in their entireties) are targets for RNAi and useful in methods of the present invention.

The model plant system of Arabidopsis genetics provides the perfect system for studying plant mutants. The tocopherol cyclase (vte1) and homogentisate prenyltransferase (vte2) mutants do not affect growth and development at normal temperatures, but exert their influence on carbon allocation in response to cold. In maize, a mutation in the tocopherol cyclase gene (sxd1 in maize) causes a constitutive translocation defect phenotype and results in dwarf plants due to the inability of the mutant maize plants to export carbon efficiently during germination and development. In crop plants of such phenotypes, or similar phenotypes that adversely affect the growth and development of the early plant, the disruption of tocopherol cyclase of homogentisate prenyltransferase genes is accomplished via inducible means (e.g., developmentally or by chemical means), thereby allowing for normal growth and development until late in the life cycle when gene inhibition is induced and carbon is re-allocated to starch and soluble sugars.

In one embodiment, the present invention provides plants that are deficient in tocopherol biosynthesis such that under low, non-freezing growth conditions carbon allocation is altered as compared to wild type plants under the same conditions. In some embodiments, said plants are Arabidopsis species, in other embodiments said plants are crop plants including but not limited to grape, sunflower, sugar beet, leek, cassaya, rice, soybean and other cultivated beans, sugar cane, maize, alfafa, vetch, wheat, coffee, cotton, Brassica napus (rapeseed) and the species of the Cuphea genus.

In one embodiment, the present invention provides methods of generating biofuels comprising providing said plants deficient in tocopherol biosynthesis, subjecting said plants to low, non-freezing temperatures such that said plant demonstrates altered carbon allocation to leaves relative to a wild type plant, and subjecting said plant to processes for generating a biofuel usable as an energy source. In some embodiments, said biofuel is ethanol.

In one embodiment, the present invention provides transgenic plants comprising heterologous nucleic acid sequences, wherein said sequence inhibits tocopherol biosynthesis. It is not intended that the present invention be limited by the nature of the inhibiting nucleic acid sequence (e.g. antisense, siRNA, etc.). For example, in one embodiment, the present invention provides transgenic plants comprising heterologous nucleic acid sequences encoding a double stranded RNA sequence, wherein said sequence inhibits tocopherol biosynthesis.

In some embodiments, said heterologous sequences are operably linked to an inducible promoter. Preferred promoters are induced by cold (including but not limited to Arabidopsis cor15a, a cold inducible promoter as well as the wheat promoter wcs120) short days (including but not limited to poplar bark storage protein promoter), or senescence (including but not limited to SAG12, an Arabidopsis senescence promoter) or during senescence (including but not limited to maize SEE promoter, a cysteine protease induced during senescence). In another embodiment, the promoter is phloem specific, including but not limited to soybean sucrose binding promoter and the rice promoter Ys1 (which is induced under iron limiting conditions).

In some embodiments, said heterologous sequences are operably linked to the same promoter, in other embodiments said sequences are separated by a loop sequence. In some embodiments, said promoter is a tissue specific promoter, while in other embodiments the promoter is an inducible promoter or a constitutive promoter. In some embodiments, said sequences are linked to separate promoters. In some embodiments, the present invention provides for vectors comprising heterologous sequences that inhibit tocopherol synthesis, in preferred embodiments the vectors comprise sequences that inhibit tocopherol cyclase and/or homogentisate phytyl transferase. In some embodiments, the present invention provides for methods of generating biofuels using the transgenic plants as described herein.

### DESCRIPTION OF THE FIGURES

Table 1 shows tocopherol content when grown under permissive conditions.

Table 2 shows tocopherol content after cold treatment.

Table 3 shows yields and abortion rates after cold treatment.

Table 4 shows pigment content after cold treatment.

Table 5 shows amino acid, nucleotide and predicted promoter binding sequence information for various tocopherol cyclase homologs.

Table 6 shows amino acid, nucleotide and predicted promoter binding sequence information for various homogentisate phytyl transferase homologs.

FIG. 1 shows the tocopherol biosynthetic pathway and vte mutations in Arabidopsis thaliana. Enzymes are indicated by black boxes and mutations by gray letters and lines. Bold arrows show the primary biosynthetic route in wild type leaves. HPP, hydroxyphenylpyruvate; GGDP, geranylgeranyl-diphosphate; PDP, phytyl-diphosphate; HGA, homogentisic acid; MPBQ, 2-methyl-6-phytyl-1,4-benzoquinol; DMPBQ, 2,3-dimethyl-6-phytyl-1,4-benzoquinol; HPPD, HPP dioxygenase; GGDR, GGDP reductase; HPT, HGA phytyltransferase; TC, tocopherol cyclase; MT, MPBQ methyltransferase; γ-TMT, γ-tocopherol methyltransferase; vte1, vte2 and vte4, mutants of TC, HPT and γ-TMT, respectively.

FIGS. 2A-E show tocopherol, lipid peroxide, anthocyanin, and photosynthetic pigment content of Col and the vte2 mutant during four weeks of low temperature treatment. Col (closed circles) and vte2-1 (open squares) were grown under permissive conditions for four weeks and transferred to 7.5° C. conditions at the beginning of the light cycle for the indicated time. Data are means±SD (n=3 or 4). Student's t-test of vte2-1 relative to Col at each time point (*P<0.05, **P<0.01). (A) Total tocopherols; (B) Lipid peroxides; (C) Total chlorophylls; (D) Anthocyanins; (E) Total carotenoids.

FIG. 3 shows photosynthetic electron transport rate (ETR) of Col and the vte2 mutant during four weeks of low temperature treatment. Col (closed circles) and vte2-1 (open squares) were grown under permissive conditions for four weeks and then transferred to 7.5° C. conditions at the beginning of the light cycle for the indicated time. Analysis was conducted in the middle of the light cycle. Data are means±SD (n=4). Student's t-test of vte2-1 relative to Col at each time point (*P<0.05).

FIGS. 4A-D show changes in starch and soluble sugar levels in Col and the vte2 mutant during four weeks of low temperature treatment. Col (closed circles) and vte2-1 (open squares) were grown under permissive conditions for four weeks and then transferred to 7.5° C. conditions at the beginning of the light cycle for the indicated time. Samples were harvested at the end of light cycles. 0 days of cold treatment indicates the end of the light cycle of the day prior to initiating 7.5° C. treatment. Starch is expressed as μmol glucose equivalents/g FW. Data are means±SD (n=3 or 4). Student's t-test of vte2-1 relative to Col at each time point (*P<0.05, **P<0.01). (A) Starch; (B) Glucose; (C) Fructose; (D) Sucrose.

FIGS. 5A-D show diurnal changes in starch and soluble sugar levels in Col and the vte2 mutant during the first four days of low temperature treatment. Col (closed circles) and vte2-1 (open squares) were grown under permissive conditions for four weeks and then transferred to 7.5° C. conditions at the beginning of the light cycle for the indicated time. Samples were harvested at the end of dark and light cycles. Gray shadows indicate 12 h dark cycles. 0 h of cold treatment indicates the beginning of the first light cycle of low temperature treatment. Starch is expressed as mol glucose equivalents/g FW. Data are means±SD (n=5). Student's t-tests of vte2-1 relative to Col at each time point (*P<0.05, **P<0.01). (A) Starch; (B) Glucose; (C) Fructose; (D) Sucrose.

FIGS. 6A-G show biochemical phenotypes in mature and young leaves of Col, and the vte2 and vte1 mutants after four weeks of low temperature treatment. Col, vte2-1 and vte1-1 mutants were grown under permissive conditions for four weeks and then transferred to 7.5° C. conditions at the beginning of the light cycle for an additional four weeks. Mature leaves (7th to 9th oldest, black bars) and young leaves (13th to 16th oldest, white bars) were harvested at the end of the light cycle for analyses in (A), (D), (E), (F) and (G). Photosynthetic parameters in (B) and (C) were measured in the middle of the light cycle. Data are means±SD (n=4 or 5). Student's t-tests of mutant leaves relative to corresponding Col young or mature leaves are indicated (*P<0.05; **P<0.01). (A) Anthocyanin content; (B) Maximum photosynthetic efficiency (Fv/Fm); (C) Photosynthetic electron transport rate (ETR); (D) Starch content expressed as ∝mol glucose equivalents/g FW; (E) to (G) Glucose, fructose and sucrose content, respectively.

FIGS. 7A-B show translocation and export of ¹⁴C labeled photoassimilates in low temperature-treated Col and the vte2 and vte1 mutants. (A) HPLC analysis of phloem exudates collected from mature leaves of Col and vte2-1 treated for 10 days at 7.5° C. The HPLC trace of sugar standards is shown as dotted grey lines. The percentage of label detected in the glucose/fructose or sucrose fractions are indicated as means±SD (n=3). Glu, glucose; Fm, fructose; Suc, sucrose; Raf, raffinose. (B) Phloem exudation of ¹⁴C labeled photoassimilates from Col and vte2-1 and vte1-1 mature leaves during 7 days of 7.5° C. treatment. Total ¹⁴C fixed per mg fresh weight of each sample at the indicated time following transfer to 7.5° C. is shown below each graph. Data are means±SD (n=6 to 8). Student's t-tests relative to Col (*P<0.05, **P<0.01). N.A., data not available.

### DEFINITIONS

As used herein, the term “host cell” refers to any cell capable of replicating and/or transcribing and/or translating a heterologous gene. Thus, a “host cell” refers to any eukaryotic or prokaryotic cell (e.g., plant cells, algal cells, bacterial cells such as yeast cells, E. coli, insect cells, etc.), whether located in vitro or in vivo. For example, a host cell may be located in a transgenic plant, or located in a plant part or part of a plant tissue or in cell culture. The terms “eukaryotic” and “eukaryote” are used in it broadest sense. It includes, but is not limited to, any organisms containing membrane bound nuclei and membrane bound organelles. Examples of eukaryotes include but are not limited to plants, yeast, animals, alga, diatoms, and fungi. The terms “prokaryote” and “prokaryotic” are used in it broadest sense. It includes, but is not limited to, any organisms without a distinct nucleus. Examples of prokaryotes include but are not limited to bacteria, blue-green algae, archaebacteria, actinomycetes and mycoplasma.

As used herein, terms defined in the singular are intended to include those terms defined in the plural and vice versa.

As used herein, the term “transgenic” when used in reference to a plant or leaf or fruit or seed for example a “transgenic plant,” transgenic leaf,” “transgenic fruit,” “transgenic seed,” or a “transgenic host cell” refers to a plant or leaf or fruit or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

As used herein, the term “transgene” refers to a foreign gene that is placed into an organism or host cell by the process of transfection. The term “foreign gene” or heterologous gene refers to any nucleic acid (e.g., gene sequence) that is introduced into the genome of an organism or tissue of an organism or a host cell by experimental manipulations, such as those described herein, and may include gene sequences found in that organism so long as the introduced gene does not reside in the same location, as does the naturally occurring gene.

As used herein, the terms “transformants” and “transformed cells” include the primary transformed cell and cultures derived from that cell without regard to the number of transfers. Resulting progeny may not be precisely identical in DNA content, due to deliberate or inadvertent mutations. Mutant progeny that have the same functionality as screened for in the originally transformed cell are included in the definition of transformants. The term “Agrobacterium” refers to a soil-borne, Gram-negative, rod-shaped phytopathogenic bacterium that causes crown gall. Agrobacterium is a representative genus of a soil-borne, Gram-negative, rod-shaped phytopathogenic bacterium family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Agrobacterium tumefaciens causes crown gall disease by transferring some of its DNA to the plant host. The transferred DNA (T-DNA) is stably integrated into the plant genome, where its expression leads to the synthesis of plant hormones and thus to the tumorous growth of the cells. A putative macromolecular complex forms in the process of T-DNA transfer out of the bacterial cell into the plant cell.

The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain GV3101, LBA4301, C58, A208, etc.) are referred to as “nopaline-type” Agrobacteria; Agrobacterium strains which cause production of octopine (e.g., strain LBA4404, Achy, B6, etc.) are referred to as “octopine-type” Agrobacteria; and Agrobacterium strains which cause production of agropine (e.g., strain EHA105, EHA101, A281, etc.) are referred to as “agropine-type” Agrobacteria.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

As used herein, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software.

The term “fusion” when used in reference to a polypeptide refers to a chimeric protein containing a protein of interest joined to an exogenous protein fragment (the fusion partner). The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms. The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

As used herein, the term “plant” is used in it broadest sense. It includes, but is not limited to, any species of grass (e.g. turf grass), ornamental or decorative, crop or cereal (e.g. maize, soybean), fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As Used herein, the term “plant part” as used herein refers to a plant structure or a plant tissue, for example, pollen, an ovule, a tissue, a pod, a seed, a leaf and a cell. Plant parts may comprise one or more of a tiller, plug, rhizome, sprig, stolen, meristem, crown, and the like. In some embodiments of the present invention transgenic plants are crop plants. The terms “crop” and “crop plant” is used herein its broadest sense. The term includes, but is not limited to, any species of plant or alga edible by humans or used as a feed for animals or fish or marine animals, or consumed by humans, or used by humans (natural pesticides), or viewed by humans (flowers) or any plant or alga used in industry or commerce or education. Indeed, a variety of crop plants are contemplated, including but not limited to soybean and other cultivated beans, barley, sorgham, rice, corn, wheat, tomato, potato, pepper, onions, Arabidopsis sp., melons, cotton, turf grass, sunflower, herbs and trees.

As used herein, the term plant cell “compartments or organelles” is used in its broadest sense. As used herein, the term includes but is not limited to, the endoplasmic reticulum, Golgi apparatus, trans Golgi network, plastids, sarcoplasmic reticulum, glyoxysomes, mitochondrial, chloroplast, thylakoid membranes and nuclear membranes, and the like.

As used herein, the term “trait” in reference to a plant refers to an observable and/measurable characteristics of an organism, such as cold tolerance in a plant or microbe. As used herein, the term “agronomic trait” and “economically significant trait” refers to any selected trait that increases the commercial value of a plant part, for example a preferred yield, a oil content, protein content, seed protein content, seed size, seed color, seed coat thickness, seed sugar content, leaf soluble sugar content, leaf starch content, seed free amino acid content, seed germination rate, seed texture, seed fiber content, food-grade quality, hilum color, seed yield, color of a plant part, drought resistance, water resistance, cold weather resistance, hot weather resistance, and growth in a particular hardiness zone.

As used herein, “aerial” and “aerial parts of Arabidopsis plants” refers to any plant part that is above water in aquatic plants or any part of a terrestrial plant part found above ground level.

The term “variety” refers to a biological classification for an intraspecific group or population, that can be distinguished from the rest of the species by any characteristic (for example morphological, physiological, cytological, etc.). A variety may originate in the wild but can also be produced through selected breeding (for example, see, cultivar). The terms “cultivar,” “cultivated variety,” and “cv” refer to a group of cultivated plants distinguished by any characteristic (for example morphological, physiological, cytological, etc.) that when reproduced sexually or asexually, retain their distinguishing features to produce a cultivated variety.

The term “propagation” refers to the process of producing new plants, either by vegetative means involving the rooting or grafting of pieces of a plant, or by sowing seeds. The terms “vegetative propagation” and “asexual reproduction” refer to the ability of plants to reproduce without sexual reproduction, by producing new plants from existing vegetative structures that are clones, i.e., plants that are identical in all attributes to the mother plant and to one another. For example, the division of a clump, rooting of proliferations, or cutting of mature crowns can produce a new plant.

The terms “tissue culture” and “micropropagation” refer to a form of asexual propagation undertaken in specialized laboratories, in which clones of plants are produced from small cell clusters from very small plant parts (e.g. buds, nodes, leaf segments, root segments, etc.), grown aseptically (free from any microorganism) in a container where the environment and nutrition can be controlled.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. The term “a nucleotide comprising at least a portion of a gene” may comprise fragments of the gene or the entire gene. The term “cDNA” refers to a nucleotide copy of the “messenger RNA” or “mRNA” for a gene. In some embodiments, cDNA is derived from the mRNA. In some embodiments, cDNA is derived from genomic sequences. In some embodiments, cDNA is derived from EST sequences. In some embodiments, cDNA is derived from assembling portions of coding regions extracted from a variety of BACs, contigs, Scaffolds and the like.

The term “gene” encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The term “gene” encompasses both cDNA and genomic forms of a gene. A genomic form or clone of a gene contains the coding region termed “exon” or “expressed regions” or “expressed sequences” interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The terms “allele” and “alleles” refer to each version of a gene for a same locus that has more than one sequence. For example, there are multiple alleles for eye color at the same locus. The terms “recessive,” “recessive gene,” and “recessive phenotype” refer to an allele that has a phenotype when two alleles for a certain locus are the same as in “homozygous” or as in “homozygote” and then partially or fully loses that phenotype when paired with a more dominant allele as when two alleles for a certain locus are different as in “heterozygous” or in “heterozygote.” The terms “dominant,” “dominant allele,” and “dominant phenotype” refer to an allele that has an effect to suppress the expression of the other allele in a heterozygous (having one dominant allele and one recessive allele) condition.

The term “heterologous” when used in reference to a gene or nucleic acid refers to a gene that has been manipulated in some way. For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The terms “nucleic acid sequence,” “nucleotide sequence of interest” or “nucleic acid sequence of interest” refer to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “structural” when used in reference to a gene or to a nucleotide or nucleic acid sequence refers to a gene and/or A nucleotide or nucleic acid sequence whose ultimate expression product is a protein (such as an enzyme or a structural protein), an rRNA, an sRNA, a tRNA, and the like.

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polynucleotide” refers to refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “an oligonucleotide (or polypeptide) having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the expression vectors of the present invention may contain endogenous enhancers, exogenous promoters, splice junctions, intervening sequences, polyadenylation signals, etc., or a combination of both endogenous and exogenous control elements.

As used herein, the term “exogenous promoter” refers to a promoter in operable combination with a coding region wherein the promoter is not the promoter naturally associated with the coding region in the genome of an organism. The promoter which is naturally associated or linked to a coding region in the genome is referred to as the “endogenous promoter” for that coding region.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “EST” and “expressed sequence tag” refer to a unique stretch of DNA within a coding region of a gene; approximately 200 to 600 base pairs in length. The term “recombinant” when made in reference to a nucleic acid molecule refers to a nucleic acid molecule that is comprised of segments of nucleic acid joined together by) means of molecular biological techniques. The term “recombinant” when made in reference to a protein or a polypeptide refers to a protein molecule that is expressed using a recombinant nucleic acid molecule.

The terms “protein,” “polypeptide,” “peptide,” “encoded product,” “amino acid sequence,” are used interchangeably to refer to compounds comprising amino acids joined via peptide bonds and a “protein” encoded by a gene is not limited to the amino acid sequence encoded by the gene, but includes post-translational modifications of the protein. Where the term “amino acid sequence” is recited herein to refer to an amino acid sequence of a protein molecule, the term “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The deduced amino acid sequence from a coding nucleic acid sequence includes sequences which are derived from the deduced amino acid sequence and modified by post-translational processing, where modifications include but not limited to glycosylation, hydroxylations, phosphorylations, and amino acid deletions, substitutions, and additions. Thus, an amino acid sequence comprising a deduced amino acid sequence is understood to include post-translational modifications of the encoded and deduced amino acid sequence. The term “X” may represent any amino acid.

The terms “homolog,” “homologue,” “homologous,” and “homology” when used in reference to amino acid sequence or nucleic acid sequence or a protein or a polypeptide refers to a degree of sequence identity to a given sequence, or to a degree of similarity between conserved regions, or to a degree of similarity between three-dimensional structures or to a degree of similarity between the active site, or to a degree of similarity between the mechanism of action, or to a degree of similarity between functions. In some embodiments, a homologue has a greater than 30% sequence identity to a given sequence. In some embodiments, a homologue has a greater than 40% sequence identity to a given sequence. In some embodiments, a homologue has a greater than 60% sequence identity to a given sequence. In some embodiments, a homologue has a greater than 70% sequence identity to a given sequence. In some embodiments, a homologue has a greater than 90% sequence identity to a given sequence. In some embodiments, a homologue has a greater than 95% sequence identity to a given sequence. In some embodiments, homology is determined by comparing internal conserved sequences to a given sequence. In some embodiments, homology is determined by comparing designated conserved functional and/or structural regions, for example a RING domain, a low complexity region or a transmembrane region.

The term “homology” when used in relation to nucleic acids or proteins refers to a degree of identity. There may be partial homology or complete homology. The following terms are used to describe the sequence relationships between two or more polynucleotides and between two or more polypeptides: “identity,” “percentage identity,” “identical,” “reference sequence,” “sequence identity,” “percentage of sequence identity,” and “substantial identity.” “Sequence identity” refers to a measure of relatedness between two or more nucleic acids or proteins, and is described as a given as a percentage “of homology” with reference to the total comparison length. A “reference sequence” is a defined sequence used as a basis for a sequence comparison; a reference sequence may be a subset of a larger sequence, for example, the sequence that forms an active site of a protein or a segment of a full-length cDNA sequence or may comprise a complete gene sequence. Since two polynucleotides or polypeptides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of in internal region of a polypeptide. In one embodiment, a comparison window is at least 77 amino acids long. In another embodiment, a comparison window is at least 84 amino acids long. In another embodiment, conserved regions of proteins are comparison windows. In a further embodiment, an amino acid sequence for a conserved transmembrane domain is 24 amino acids. Calculations of identity-may be performed by algorithms contained within computer programs such as the ClustalX algorithm (Thompson et al. 1997, Nucleic Acids Res. 24:4876-4882), herein incorporated by reference; MEGA2 (version 2.1) (Kumar et al., 2001, Bioinformatics 17:1244-1245); “GAP” (Genetics Computer Group, Madison, Wis.), “ALIGN” (DNAStar, Madison, Wis.), BLAST (National Center for Biotechnology Information; NCBI as described at http://www.ncbi.nlm.nih.gov/BLAST/blast_help.shtml) and MultAlin (Multiple sequence alignment) program (Corpet, 1988, Nucl. Acids Res., 16: 10881-10890) at http://prodes.toulouse.inra.fr/multalin/multalin.html), all of which are herein incorporated by reference).

The term “sequence identity” means that two polynucleotide or two polypeptide sequences are identical (i.e., on a nucleotide-by-nucleotide basis or amino acid basis) over the window of comparison. The term “percentage of sequence identity” is calculated by comparing two optimally aligned sequences over the window of comparison, determining the number of positions at which the identical nucleic acid base (e.g., A, T, C, G, U, or I) or amino acid, in which often conserved amino acids are taken into account, occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present.

The term “ortholog” refers to a gene in different species that evolved from a common ancestral gene by speciation. In some embodiments, orthologs retain the same function. The term “paralog” refers to genes related by duplication within a genome. In some embodiments, paralogs evolve new functions. In further embodiments, a new function of a paralog is related to the original function.

The term “partially homologous nucleic acid sequence” refers to a sequence that at least partially inhibits (or competes with) a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely complementary to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial-degree of identity (e.g., less than about 30% identity); in the absence of non-specific binding the probe will not hybridize to the second non-identical target.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “vector” refers to nucleic acid molecules that transfer DNA segment(s). Transfer can be into a cell, cell to cell, etc. The term “vehicle” is sometimes used interchangeably with “vector.”

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. The term “expression vector” when used in reference to a construct refers to an expression vector construct comprising, for example, a heterologous DNA encoding a gene of interest and the various regulatory elements that facilitate the production of the particular protein of interest in the target cells. In certain embodiments of the present invention, a nucleic acid sequence of the present invention within an expression vector is operatively linked to an appropriate expression control sequence(s) (promoter) to direct mRNA synthesis.

The terms “in operable combination,” “in operable order,” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, and the like.

Transcriptional control signals in eukaryotes comprise “promoter” and “enhancer” elements. Promoters and enhancers consist of short arrays of DNA sequences that interact specifically with cellular proteins involved in transcription (Maniatis et al., 1987, Science 236:1237; herein incorporated by reference). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types.

The terms “promoter element,” “promoter,” or “promoter sequence” refer to a DNA sequence that is located at the 5′ end (i.e. precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer, and is typically between about 500 by and 4 kb in length, and is preferably about 1 to 1.5 kb in length. Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene and/or A reporter gene expressing a reporter molecule, to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be “constitutive” or “inducible.” The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098, herein incorporated by reference), and ubi3 promoters (see e.g., Garbarino and Belknap, 1994, Plant Mol. Biol. 24:119-127, herein incorporated by reference). Such promoters have been used successfully to direct the expression of heterologous nucleic acid sequences in transformed plant tissue.

In contrast, an “inducible” promoter is one that is capable of directing a level of transcription of an operably linked nucleic acid sequence in the presence of a stimulus (e.g., heat shock, chemicals, light, etc.) that is different from the level of transcription of the operably linked nucleic acid sequence in the absence of the stimulus.

The term “regulatory element” refers to a genetic element that controls some aspect of the expression of nucleic acid sequence(s). For example, a promoter is a regulatory element that facilitates the initiation of transcription of an operably linked coding region. Other regulatory elements are splicing signals, polyadenylation signals, termination signals, and the like.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

The term “naturally linked” or “naturally located” when used in reference to the relative positions of nucleic acid sequences means that the nucleic acid sequences exist in nature in the relative positions.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al. Molecular Cloning: A Laboratory Manual, 2.sup.nd ed., Cold Spring Harbor Laboratory Press, New York (1989) pp. 16.7-16.8, herein incorporated by reference). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The terms “stable transfection” and “stably transfected” refer to the introduction and integration of foreign DNA into the genome of the transfected cell. The term “stable transfectant” refers to a cell that has stably integrated foreign DNA into the genomic DNA.

The terms “transient transfection” and “transiently transfected” refer to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes.

The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb in Virol., 52:456 (1973), herein incorporated by reference, has been modified by several groups to optimize conditions for particular types of cells. The art is well aware of these numerous modifications.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment, and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, herein incorporated by reference), and are commercially available (e.g. the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al. Mol. Cell. Biol. 7:725 (1987) and U.S. Pat. Nos. 6,074,859; 5,976,796; 5,674,713; and 5,618,682; all of which are herein incorporated by reference), green fluorescent protein (e.g., GenBank Accession Number U43284; GFP variants commercially available from CLONTECH Laboratories, Palo Alto, Calif., herein incorporated by reference), chloramphenicol acetyltransferase, β-galactosidase (lacZ gene), alkaline phosphatase, and horse radish peroxidase.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Carthew (2001) has reported (Curr. Opin. Cell Biol. 13(2):244-248) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The terms “hpRNA” and “hairpin RNA” refer to self-complementary RNA that forms hairpin loops and functions to silence genes (e.g. Wesley et al. (2001) The Plant Journal 27(6):581-590 herein incorporated by reference). The term “ihpRNA” refers to intron-spliced hpRNA that functions to silence genes.

The term “target RNA molecule” refers to an RNA molecule to which at least one strand of the short double-stranded region of a siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The terms “posttranscriptional gene silencing” and “PTGS” refers to silencing of gene expression in plants after transcription, and appears to involve the specific degradation of mRNAs synthesized from gene repeats. The term “cosuppression” refers to silencing of endogenous genes by heterologous genes that share sequence identity with endogenous genes.

The term “coexpression” refers to the expression of a foreign gene that has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

The term “overexpression” generally refers to the production of a gene product in transgenic organisms that exceeds levels of production in normal or non-transformed organisms.

The terms “overexpression” and “overexpressing” and grammatical equivalents, are specifically used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis.

The term “isolated” when used in relation to a nucleic acid or polypeptide, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences that are removed from their natural environment isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the terms “purified” and “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue, such as a leaf. In another sense, it is meant to include a specimen or culture obtained from any source, as well as biological and environmental samples. Biological samples may be obtained from plants or animals and encompass fluids, solids, tissues, and gases. Environmental samples include environmental material such as surface matter, soil, water, salt, and industrial samples. These examples are not to be construed as limiting the sample types applicable to the present invention.

### DETAILED DESCRIPTION OF THE INVENTION

Tocopherols (Vitamin E), lipid soluble antioxidants only found in photosynthetic organisms, have long been assumed to play essential roles in protecting plants from oxidative stress. There are four known tocopherols (α, β, γ and δ) all consisting of a chromanol head group attached to a phytyl tail, differeing only in the number and position of methyl groups on the chromanol ring. Tocopherols are amphiphatic molecules that form complexes with specific lipid constituents and physically stabilize membranes (Wassail et al., 1986, Biochem. 25:319-326; Stillwell et al., 1996, Biochem. 35:13353-13362; Wang and Quinn, 2000, Mol. Membr. Biol. 17:143-156; Bradford et al., 2003, J. Lipid Res. 44:1940-1945). Tocopherols can efficiently quench singlet oxygen, scavenge various radicals, and thereby terminate lipid peroxidation chain reactions (Liebler and Burr, 1992, Biochem. 31:8278-8284; Bramley et al., 2000, J. Food Sci. Agric. 80:913-938; Schneider, 2005, Mol. Nutr. Food Res. 49:7-30).

In plants, tocopherols are synthesized and localized in plastid membranes that are also highly enriched in polyunsaturated fatty acids (PUFA) (Bucke, 1968, Phytochemistry 7:693-700; Soll et al., 1980, FEBS Lett. 112:243-246; Lichtenthaler et al., 1981, Biochim. Biophys. Acta. 641:99-105; Soll et al., 1985, Arch. Biochem. Biophys. 238:290-299; Soll, 1987, Meth. Enzym. 148:383-392; Vidi et al., 2006, J. Biol. Chem. 281:11225-11234), and increased tocopherol content has been correlated in the response of photosynthetic tissues to a variety of abiotic stresses, including high light intensity (HL), salinity, drought and low temperatures (Munne-Bosch et al., 1999, Plant Physiol. 121:1047-1052; Keles and Oncel, 2002, Plant Sci. 163:783-790; Bergmuller et al., 2003, Plant Mol. Biol. 52:1181-1190; Collakova and DellaPenna, 2003, Plant Physiol. 133:930-940). The present invention is not limited to a particular mechanism. Indeed, an understanding of the mechanism is not necessary to practice the present invention. Nonetheless, it is contemplated that a primary function of tocopherols in plants is to protect photosynthetic membranes from oxidative stresses by acting as lipid-soluble antioxidants. Therefore, the use of transgenic plants (e.g., Arabadopsis sp.) that exhibit disrupted tocopherol biosynthetic pathways are useful tools to directly investigate tocopherol functions in plants. Such mutants are useful in methods of the present invention.

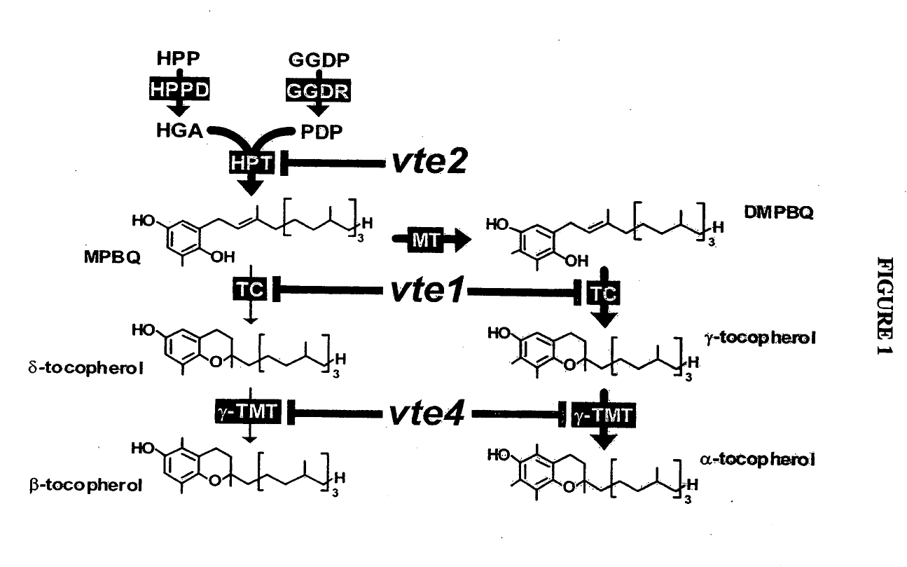

A mutant plant that contains a defective homogentisate phytyl transferase gene (HPT, also known as Vitamin E2, vte2) (for example, see Genbank Accession No. NM_{—}179653, incorporated herein in its entirety) lacks all tocopherols and pathway intermediates (FIG. 1, Table 1). vte2 mutants are severely impaired in seed longevity and early seedling development due to the massive and uncontrolled peroxidation of storage lipids (Sattler et al., 2004, Plant Cell 16:1419-1432; Sattler et al., 2006, in submission), consistent with loss of the lipid soluble antioxidant functions of tocopherols (Ham and Liebler, 1995, Biochem. 34:5754-5761; Ham and Liebler, 1997, Arch. Biochem. Biophys. 339:157-164). The severe impact of tocopherol deficiency on seedling fitness provides a strong selective pressure for retention of tocopherol synthesis during seed plant evolution. Interestingly, the vte2 mutants that survive early seedling development become virtually indistinguishable from wild type plants under standard growing conditions (Sattler 2004; Maeda et al., 2006, in submission). The present invention is not limited to a particular mechanism. Indeed, an understanding of the mechanism is not necessary to practice the present invention. Nonetheless, it is contemplate that unlike seed dormancy and germination, tocopherols are dispensable in mature plants in the absence of stress. Consistent with this, constitutive over-expression of vte2 in Arabidopsis demonstrated increased total leaf tocopherols, but had no discernable effect relative to wild type on plant growth or chlorophyll and carotenoid content in the absence of stress or under combined nutrient and HL stress (Collakova and DellaPenna, 2003).

A mutant plant that contains a defective tocopherol cyclase gene activity (vte1) (for example, see Genbank Accession No. NM_{—}119530, incorporated herein in its entirety) and deficient in all tocopherols is unlike the vte2 in that it accumulates the redox active biosynthetic intermediate 2,3-dimethyl-6-phytyl-1,4-benzoquinol (DMPBQ) (FIG. 1, Table 1). When grown at 100-120 μmol photon/m²/s, vte1 plants are virtually identical to wild type plants at all developmental stages (Porfirova et al., 2002, Proc. Natl. Acad. Sci. 99:12495-12500; Sattler et al., 2003, Plant Physiol. 132:2184-2195; Sattler et al., 2004). The lipid peroxidation phenotype observed in germinating vte2 seedlings is completely attenuated in vte1 plants, indicating that DMPBQ can fully compensate for tocopherols as a lipid soluble antioxidant in seedlings (Sattler et al., 2004). Under HL stress (5 days at 850 μmol photon/m²/s; Porfirova et al., 2002) or a combination of low temperature and HL stress (5 days at 6-8° C. and 1100 μmol photon/m²/s; Havaux et al., 2005, Plant Cell 17:3451-3469), vte1 plants were nearly identical to wild type plants for all parameters measured, including lipid peroxidation, with the exception of a slight decrease in maximum photosynthetic efficiency (Fv/Fm). Only under extreme conditions (24 hrs at 3° C. and continuous 1500-1600 μmol photon/m²/s) did vte1 show a rapid induction of lipid peroxidation than wild type, although this difference was transient and after 48 hours of treatment lipid peroxidation was similarly elevated in vte1 and wild type plants (Havaux et al., 2005). The present invention is not limited to a particular mechanism. Indeed, an understanding of the mechanism is not necessary to practice the present invention. Nonetheless, it is contemplated that the primary function of tocopherols in to control non-enzymatic lipid oxidation, especially during seed storage and early germination and photosynthetic tissues, but only under extreme combinations of HL and low temperature stresses.

A maize tocopherol cyclase mutant (sxd1, sucrose export defective 1) was identified prior to the identification of Arabidopsis vte1, not due to its impact on tocopherol synthesis, but because of accumulation of carbohydrates and anthocyanins in sxd1 source leaves that coincided with aberrant plasmodesmata between the bundle sheath and vascular parenchyma cells (Russin et al., 1996, Plant Cell 8:645-658; incorporated herein in its entirety). Cloning of the sxd1 locus did not provide insight into the biochemical activity of the nuclear encoded chloroplast localized protein (Provencher et al., 2001, Plant Cell 13:1127-1141, incorporated herein in its entirety) and it is only in retrospect that sxd1 is demonstrated to have tocopherol cyclase activity (Sattler et al., 2003). The maize sxd1 carbohydrate accumulation phenotype was intriguing as it suggested an unexpected link between the tocopherol pathway and primary carbohydrate metabolism, though the mechanism involved was unclear. A similar carbohydrate phenotype did not occur in the orthologous Arabidopsis vte1 mutant (Sattler et al., 2003) but was observed in vte1 RNAi knockdown lines in potato (Hofius et al., 2004, Plant Physiol. 135:1256-1268, incorporated herein in its entirety).

It is contemplated that in contrast to long held assumptions about tocopherol functions in plants, tocopherol deficient mutants are remarkably similar to wild type in their response to abiotic stresses with the notable exception of increased sensitivity to non-freezing low temperatures. It is demonstrated herein that physiological, biochemical and ultrastructural data demonstrate that the earliest impact of tocopherol deficiency during low temperature treatment is an inhibition of photoassimilate transport associated with structural changes in phloem parenchyma transfer cells, a bottleneck for photoassimilate transport. The resulting accumulation of carbohydrates in source leaves impacts the physiology and response of the entire plant to low temperature.

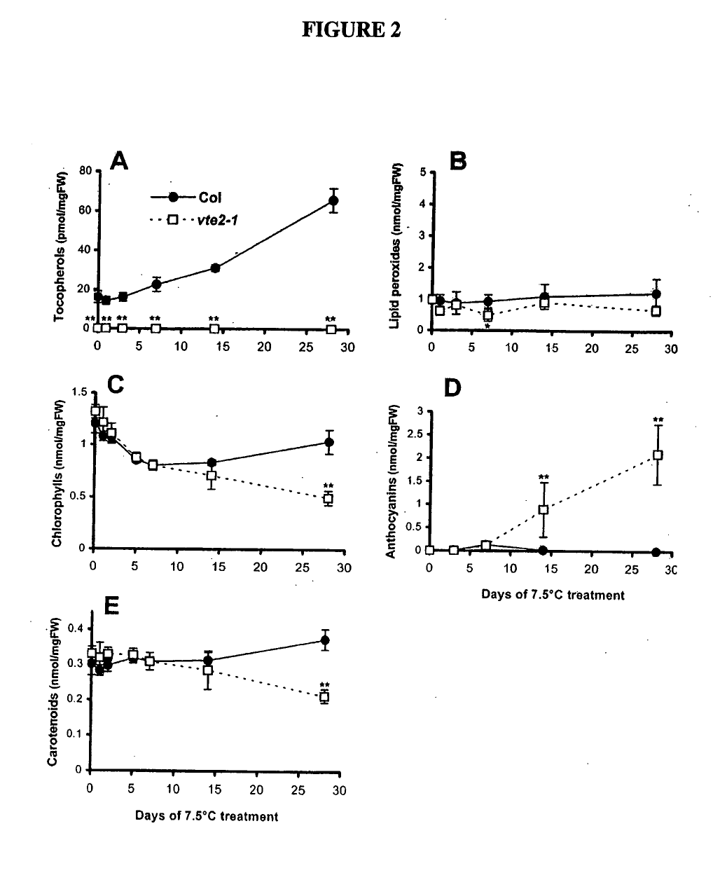

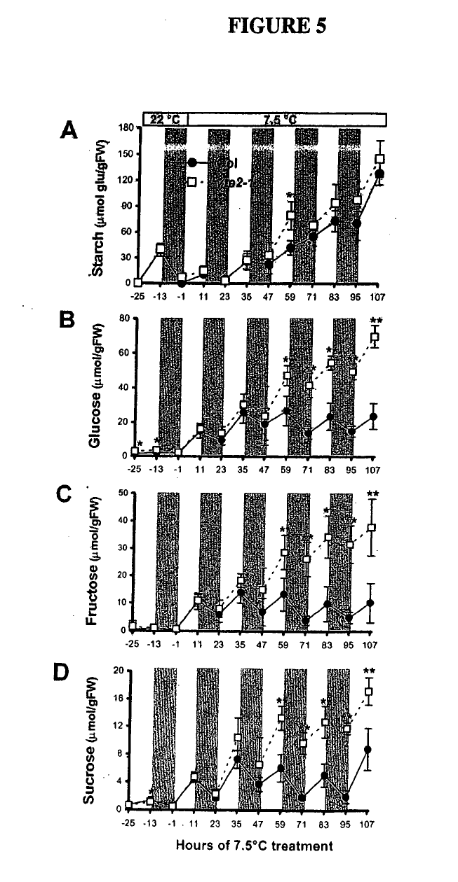

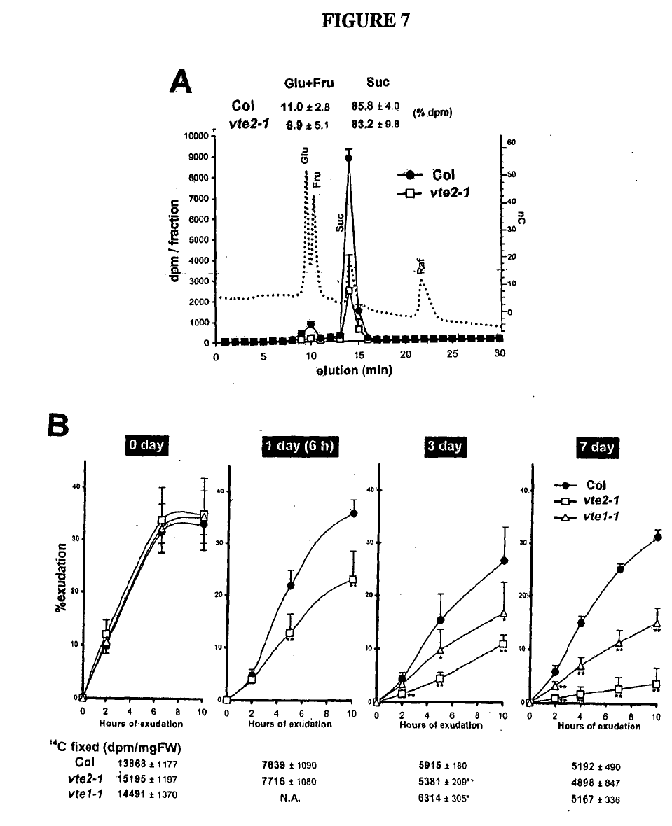

A response of plants to low temperatures is the accumulation of soluble sugars and other osmoprotectants, which are critical components for the process of cold acclimation leading to freezing tolerance (Wanner and Junttila, 1999, Plant Physiol. 120:391-399; Gilmour et al., 2000, Plant Physiol. 124:1854-1865, incorporated herein in their entireties). The subsequent recovery of photosynthesis and sucrose metabolism is an important component of low temperature adaptation in that it provides carbon to sustain growth under low temperatures (Strand et al., 1997, Plant J. 12:605-614; Strand et al., 1999, Plant Physiol. 119:1387-1398, incorporated herein in their entireties). During the first two days of low temperature treatment, soluble sugar levels increased similarly in both vte2 and wild type (FIG. 5), suggesting tocopherols have little impact on the initial accumulation of soluble sugars in response to low temperature. However, the accumulation of sucrose and other soluble sugars was much higher in vte2 than wild type after 60 h of low temperature treatment (FIGS. 5B-D), although the rates of photosynthesis and carbon fixation were indistinguishable between the two genotypes until 14 days at low temperature (FIGS. 2B and 7B). vte2 also reduced soluble sugar levels more slowly at night than wild type after 3 days of low temperature treatment (FIGS. 5B-D). These results suggest that tocopherol deficiency affects carbohydrate utilization/mobilization rather than the supply of fixed carbon from photosynthesis during low temperature adaptation. ¹⁴CO₂-labeling experiments demonstrated that in comparison to wild type, low temperature-treated vte2 translocated significantly less ¹⁴C-labeled photoassimilates from leaves (source tissue) to roots (sink tissue). The long distance transport of photoassimilates occurs through phloem and the transport rate is determined either by the rate of export from source leaves to phloem (loading) or by removal into sink tissues (unloading) (Vanbel, 1993; Stitt, 1996, Plant Cell 8:565-571; Herbers and Sonnewald, 1998, Curr. Opin. Plant Biol. 1:207-216, incorporated herein in their entireties).

Phloem exudation experiments with excised leaves showed that vte2 source leaves exported significantly less ¹⁴C labeled photoassimilates than wild type as early as 6 h following transfer to low temperature (FIG. 7B). The rapidity of this reduction in photoassimilate export in comparison to the elevated sugar accumulation in vte2 starting at 60 h indicates that impaired photoassimilate export is an early, upstream event in the vte2 low temperature phenotype and it is contemplated to be the root cause of the elevated sugar accumulation in low temperature-treated vte2. Taken together, these analyses demonstrate that tocopherols are required for proper regulation of photoassimilate export from source leaves and thereby play a critical role in low temperature adaptation in Arabidopsis. Previous studies of the maize sxd1 mutant and potato vte1-RNAi lines (both affecting tocopherol cyclase activity) suggests a linkage between carbohydrate metabolism and tocopherol biosynthesis, as in both cases carbohydrates accumulated to high levels in mature leaves at normal growth temperatures (19 to 30° C., Russin et al., 1996; Provencher et al., 2001; Hofius et al., 2004).

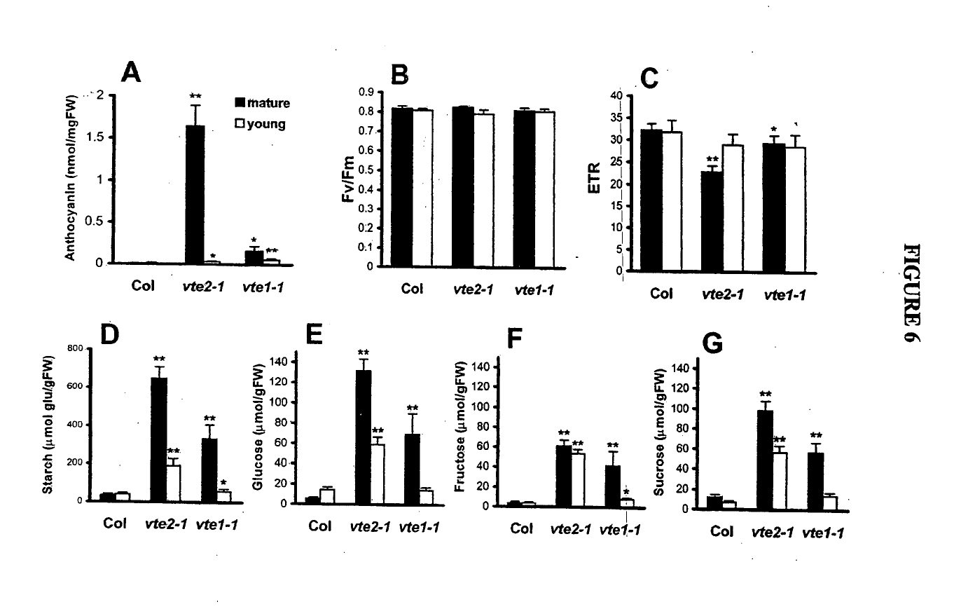

Tocopherol-deficient Arabidopsis mutants exhibit a phenotype that is analogous to sxd1 but which is inducible only at low temperatures. Thus, the linkage between tocopherol biosynthesis and carbohydrate metabolism is conserved among all tocopherol-deficient mutants identified in higher plants to date (maize sxd1, potato vte1-RNAi and low temperature-treated Arabidopsis vte1 and vte2). Although the maize sxd1 mutant and potato vte1-RNAi line suggested tocopherol chromanol ring cyclization was somehow related to regulation of carbohydrate metabolism, it was unclear whether the phenotype was due to the lack of tocopherols or accumulation of the redox active quinol intermediate DMPBQ (Sattler et al., 2003; Hofius et al., 2004). Analysis of the full suite of Arabidopsis vte mutants allows a conclusive answer to this question. Given that vte2 lacks DMPBQ (Table 1) and exhibits a more severe carbohydrate accumulation phenotype than vte1 (FIG. 6), it is contemplated that it is the absence of tocopherols rather than accumulation of DMPBQ that causes the carbohydrate accumulation phenotype. It is contemplated that the reduced severity of the carbohydrate accumulation phenotype in vte1 suggests that DMPBQ partially suppresses the low temperature-inducible vte2 carbohydrate accumulation phenotype.

The carbohydrate accumulation phenotype of maize sxd1 was reported to be associated with altered structural features within vascular tissue. Plasmodesmata at the sxd1 bundle sheath/vascular parenchyma boundary were reported occluded by wall materials (Russin et al., 1996; Provencher et al., 2001) and subsequently suggested to correspond to aniline blue-positive fluorescence (Botha et al., 2000). This structural aberration in sxd1 plasmodesmata was posited to be the basis of the sxd1 carbohydrate accumulation phenotype because it would lead to a block in the symplastic movement of photoassimilate. Callose was also observed in vascular tissue of potato vte1-RNAi plants by light microscopy with monoclonal antibodies against β-1,3 glucan (Hofius et al., 2004). In the absence of high-resolution microscopy, Hofius et al. (2004) also suggested this vascular-associated callose somehow interrupts photoassimilate transport. However, in both the sxd1 and potato vte1-RNAi studies it was not determined whether callose deposition was a cause or effect of carbohydrate accumulation. A critical observation in the present application is that the low temperature-inducible photoassimilate export defect in Arabidopsis tocopherol-deficient mutants is temporally associated with callose deposition in a specific vascular tissue cell type.

The results are significant as they provide a direct link between defective photoassimilate export and callose deposition (or events tightly associated with callose deposition) in tocopherol-deficient mutants and exclude the possibility that callose deposition is a secondary effect caused by carbohydrate accumulation. The low temperature-inducible callose deposition in Arabidopsis vte2 selectively occurred in phloem parenchyma transfer cells. Importantly, initial callose deposition was site specific within these cells and resulted in a callose boundary between the phloem parenchyma transfer cell and sieve element/companion cell complex where transfer cell wall ingrowths occur. No evidence of callose deposition or occlusion of plasmodesmata at the bundle sheath-vascular parenchyma boundary during induction of the export defective phenotype in vte2 was seen. However, by 14 days of low temperature treatment, when vte2 contains high levels of starch and anthocyanins and more closely resembles the phenotype of maize sxd1, the entire parenchyma transfer cell became encased in a callose sheath associated with abnormally shaped transfer cell wall ingrowths and it is at this point that callose deposition is also observed in vte2 plasmodesmata at the bundle sheath-vascular parenchyma boundary.

When a comparison of the development, polarity and morphology of transfer cell walls in 7.5° C.-treated vte2 with Col is performed, it becomes clear that tocopherols play an important role in transfer cell wall synthesis at low temperatures. Previous structural studies on the minor vein structure of Arabidopsis have suggested that phloem parenchyma transfer cells are the site of apoplastic unloading of photoassimilates arriving symplastically from bundle sheath cells (Haritatos et al., 2000). The coincidence in reduction of photoassimilate export with callose deposition in these spatially distinct subcellular sites in the vte2 mutant during low temperature treatment provides direct support for the role of transfer cells in photoassimilate export from source leaves via delivery to the phloem apoplast. This callose deposition (or events associated with the callose deposition) in phloem parenchyma transfer cells of 7.5° C.-treated vte2 form a barrier to symplast-to-apoplast but not symplast-to-symplast transport. It is contemplated that the limited export that still occurs in 7.5° C.-treated vte2 source leaves is due to apoplastic unloading from bundle sheath cells and subsequent loading to the sieve element/companion cell complex.

Tocopherol-deficient vte2 mutant is remarkably similar to wild type in its response to most abiotic stresses with the notable exception of non-freezing low temperature treatments. Tocopherol-deficiency specifically results in abnormal phloem parenchyma transfer cell wall development at low temperature. This leads to rapid impairment of photoassimilate export that profoundly impacts cellular metabolism and whole plant physiology during both short and long term low temperature treatments. As this occurs in both vte2 and vte1, it is contemplated that tocopherols play a previously unrecognized role in low temperature adaptation, specifically in phloem loading. Tocopherols have important function(s) in regulating the response of these cell types to environmental stress, such as low temperatures. Photooxidative damage and photoinhibition are not associated with the vte2 low temperature phenotype and HL1800 (which approaches full sunlight) at 22° C. has little impact on vte2 compared to wild type suggesting a more limited role for tocopherols in protecting plants from photooxidative stress than has been assumed. This seems in direct contradiction with a recent report using Arabidopsis vte mutants that concluded tocopherols protect Arabidopsis against photoinhibition and photooxidative stress (Havaux et al., 2005).

In one embodiment, the present invention relates to plants with altered carbon allocation. In some embodiments, said plants are transgenic plants that demonstrate altered tocopherol biosynthesis pathways. In some embodiments, the transgenic plants are species of Arabidopsis or Brassica napus (rapeseed). In some embodiments, the transgenic plants are crop plants (e.g., maize, soybean, wheat, amaranth, etc.). In some embodiments, the transgenic plants are animal forage plants (e.g., alfalfa, vetch, etc.). In some embodiments, the Arabidopsis species that demonstrate altered tocopherol biosynthesis pathways are mutants in vte1 and vte2. In some embodiments, the crop plant maize that demonstrates altered tocopherol biosynthesis is sxd1 mutant. In some embodiments, the carbon allocation demonstrated by altered tocopherol biosynthesis results in an increase in soluble sugars and starch accumulation in the leaves of a plant as compared to a wild type plant. In some embodiments, the carbon allocation is demonstrated in a plant with altered tocopherol biosynthesis at low, non-freezing temperatures, for example, at least 3° C., at least 4° C., at least 5° C., at least 6° C., at least 7° C., at least 8° C., at least 9° C., at least 12° C. In some embodiments, the non-freezing temperature is preferably around 7.5° C.

In one embodiment, the present invention relates to methods of using plants demonstrating altered tocopherol biosythesis in the creation of biofuels. In some embodiments, the plants demonstrating altered tocopherol biosynthesis are Arabidopsis species. In some embodiments, the plants demonstrating altered tocopherol biosynthesis are crop plants, including but not limited to, grape, sunflower, sugar beet, leek, cassaya, rice, soybean and other cultivated beans, sugar cane, maize, alfafa, vetch, wheat, coffee and cotton. In some embodiments, the altered tocopherol biosynthesis is such that carbon allocation in a mutant plant is preferential to carbon allocation in leaves as soluble sugars and starch over other plant locations when compared to wild types plants. In some embodiments, the leaves with an increased amount of soluble sugars and starch when compared to wild type leaves are used in the creation of biofuels. In some embodiments, the biofuel is ethanol, or an equivalent fuel useful as an alternative alone, or in combination with, petroleum based products. In some embodiments, the biofuel is ethanol of a type useful in powering transportation vehicles and machinery that typically requires a combustible power source for power generation.

In one embodiment, the biofuel created from the mutant plants as described herein is mixed with petroleum based products thereby creating a fuel that is at least 5%, at least 10%, at least 20%, at least 40%, at least 80% biofuel that is amenable to configurations for generating power for transportation vehicles and machinery that typically requires a combustible power source. In some embodiments, the biofuel that is mixed with a petroleum based product is ethanol.

In one embodiment, the biofuel created using the compositions and methods of the present application increases the amount of biofuel obtainable from plant materials over that obtainable without using the compositions and methods of the present application. In some embodiments, the compositions and methods of the present application provide for a more cost effective way of producing biofuels over what is currently available. For example, sugar feedstocks currently used for ethanol generation include plant materials high in sugars such as sugarcane, sugar beet, sweet sorghum, and various fruits. However, these materials are generally too expensive in the United States to use for fuel ethanol production due to their uses in human consumption. Therefore, compositions and methods of the present application provide for a biofuel feedstock that is high in soluble sugars and starches and a more cost effective feedstock that is not typically used for human consumption as compared to the currently available sources.

RNAi refers to the introduction of homologous double stranded RNA (dsRNA) to target a specific gene product, resulting in post transcriptional silencing of that gene. This phenomena was first reported in Caenorhabditis elegans by Guo and Kemphues (Par-1, A gene required for establishing polarity in C. elegans embryos, encodes a putative Ser/Thr kinase that is asymmetrically distributed, 1995, Cell, 81 (4) 611-620) and subsequently Fire et al., 1998, Nature 391: 806-811 discovered that it is the presence of dsRNA, formed from the annealing of sense and antisense strands present in the in vitro RNA preps, that is responsible for producing the interfering activity.

The present invention contemplates the use of RNA interference (RNAi) to downregulate the expression of genes needed for tocopherol biosynthesis synthesis (e.g., tocopherol cyclase, homogentisate phytyl transferase). In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew has reported (Curr. Opin. Cell Biol. 13(2):244-248 (2001) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

In preferred embodiments, the dsRNA used to initiate RNAi, may be isolated from native sourcse or produced by known means, e.g., transcribed from DNA. The promoters and vectors described in more detail below are suitable for producing dsRNA. RNA is synthesized either in vivo or in vitro. In some embodiments, endogenous RNA polymerase of the cell may mediate transcription in vivo, or cloned RNA polymerase can be used for transcription in vivo or in vitro. In other embodiments, the RNA is provided by transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the dsRNA is transcribed from the vectors as two separate stands. In other embodiments, the two strands of DNA used to form the dsRNA may belong to the same or two different duplexes in which they each form with a DNA strand of at least partially complementary sequence. When the dsRNA is thus produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, and which can join in pairs to form a dsRNA. See, e.g., U.S. Pat. No. 5,795,715, incorporated herein by reference. RNA duplex formation may be initiated either inside or outside the cell.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases.

There is no upper limit on the length of the dsRNA that can be used. For example, the dsRNA can range from about 21 base pairs (bp) of the gene to the full length of the gene or more. In one embodiment, the dsRNA used in the methods of the present invention is about 1000 by in length. In another embodiment, the dsRNA is about 500 by in length. In yet another embodiment, the dsRNA is about 22 by in length. In some preferred embodiments, the sequences that mediate RNAi are from about 21 to about 23 nucleotides. That is, the isolated RNAs of the present invention mediate degradation of the target RNA (e.g., tocopherol cyclase, homogentisate phytyl transferase).

The double stranded RNA of the present invention need only be sufficiently similar to natural RNA that it has the ability to mediate RNAi for the target RNA. In one embodiment, the present invention relates to RNA molecules of varying lengths that direct cleavage of specific mRNA to which their sequence corresponds. It is not necessary that there be perfect correspondence of the sequences, but the correspondence must be sufficient to enable the RNA to direct RNAi cleavage of the target mRNA. In a particular embodiment, the RNA molecules of the present invention comprise a 3′ hydroxyl group. In some embodiments, the amount of target RNA (mRNA) is reduced in the cells of the target organism (e.g., Arabidopsis sp., maize) exposed to target specific double stranded RNA as compared to target organisms that have not been exposed to target specific double stranded RNA.

In one embodiment, the present invention utilizes RNAi genes encoding dsRNA sequences that target tocopherol biosythesis genes. In some embodiments, the RNAi sequences target plant tocopherol cyclase and or homogentisate phytyl transferase. In some embodiments, the tocopherol biosynthesis genes targeted are found in Arapbidopsis species and/or crop plants.

In some embodiments, the present invention provides transgenic plants that express dsRNA molecules that correspond to target molecules in desired plant species (e.g, Arabidopsis, crop plant species). As noted above, it is not intended that the present invention be limited by the nature of the inhibiting nucleic acid. RNA interference (RNAi) by double stranded RNA (dsRNAs) molecules of approximately 20-25 nucleotides termed short interfering (siRNAs) is a powerful method for preventing the expression of a particular gene. The dsRNA dominantly silences gene expression in a sequence-specific manner by causing the corresponding endogenous mRNA to be degraded. The technique has been applied to a wide range of organisms. Methods for expressing siRNAs in cells in culture and in vivo using viral vectors, and for transfecting cells with synthetic siRNAs, have been developed and are being used to establish the functions of specific proteins in various cell types and organisms. For example, chemically synthesized or in vitro transcribed siRNAs can be transfected into cells, injected into mice, or introduced into plants. siRNAs can also be expressed endogenously from siRNA expression vectors or PCR products in cells or in transgenic animals.

siRNA design for post-transcriptional gene silencing or RNAi is well known. Many different algorithms and programs are available for siRNA design. To design a siRNA, an mRNA or cDNA sequence of the gene of interest is required and can be retrieved from the National Center for Biotechnology Information (NCBI) database, available at www.ncbi.nlm.nih.gov, hereby incorporated by reference. The RefSeq mRNA sequence for the target gene is preferred because it represents the most reliable mRNA sequence for the gene. Many programs operate using a RefSeq ID as a sequence input, which usually looks like ‘NM_xxxxxx’. The whole length of an mRNA sequence can be targeted although some researchers prefer targeting only the coding region while excluding untranslated regions (UTR). Some programs provide homology search for candidate siRNA targets against other mRNA sequences of the genome. A siRNA should not have significant homology with unrelated sequences especially at its 3′ end. If a siRNA design program does not provide the function for homology sequence, a manual BLAST search is mandatory for avoiding off-target effects.

As described in Nature, 391: 806-811 (1998), hereby incorporated by reference, introducing double stranded RNA (dsRNA) into a cell results in potent and specific interference with expression of endogenous genes in the cell and which interference is substantially more effective than providing an individual RNA strand as proposed in antisense technology. U.S. Pat. No. 7,005,423, hereby incorporated by reference, provides methods and compositions utilizing dsRNA for targeted inhibition and/or silencing of genes of interest.

U.S. Patent Application Number 2005/0203047, hereby incorporated by reference, provides methods and compositions directed to the synthesis and use of small interfering RNA (siRNA) and micro-RNAs (miRNA), both of which are classes of dsRNAs. It has been shown that siRNA-like gene silencing mechanisms, also referred to as post-transcriptional gene silencing, can be functional in virtually any species. Many of these miRNA sequences and their position in genomes or chromosomes have been identified and are known to those skilled in the art. Until recently, when siRNA mechanisms were discovered, antisense RNA (asRNA) was the preferred method to down-regulate genes for functional studies such as target validation and pathway analysis. However, design (sequence choice), synthesis of the RNA molecules, delivery and regulation, prevented asRNA to being used significantly beyond in vitro studies. Antisense RNA in principle, and siRNA and miRNA can be expressed from gene constructs, but only siRNA and miRNA molecules are also encoded naturally in the genomes of many species. Antisense RNA usually comprises a synthetic single-stranded nucleic acid that can bind to its target by forming base pairs. Although there are some differences in the expression and maturation of siRNAs and miRNAs, the final and active product is in both cases a short, 19-22 nucleotide long, double-stranded RNA molecule as described in Dykxhoorn et al. Nature Reviews Mol. Cell. Biol., 4: 457-466 (2003) and Steinberg, Scientist, 16: 22-24 (2003), both of which are incorporated by reference. Active siRNA is typically formed by two 21-23 nucleotide long ribo-oligonucleotides that form a 19 base pair long duplex with symmetric 2-3 nucleotide long terminal overhangs having 5′ phosphate and 3′ hydroxyl groups. As a result of nucleotide sequence homology of siRNA to their cellular target RNA, one strand, usually the antisense strand binds to the target and renders it inactive and ‘flagged’ for degradation. Thus, in theory, as is the case for antisenseRNA, the antisense strand of siRNA and mciroRNA is sufficient for an inhibitory activity, but the double-stranded nature may be necessary for stability and cellular transport. Eukaryotes such as worms, plants and fungi can replicate siRNA molecules.

Although naturally occurring siRNA was detected more than five years ago, only recently have researchers attempted to apply this mechanism of action in genomic research. siRNAs can be engineered to bind or recognize virtually any RNA target in the cell and thus be used as “knock-down” tools to silence or down-regulate gene expression through RNA inactivation. The same is becoming obvious for miRNAs. Since its discovery, “asRNA” or antisense oligonucleotides are usually synthesized as deoxy-oligonucleotides or derivatives thereof as described by Zemecnik et al. Proc. Natl. Acad. Sci. USA, 75:280-284 (1978), hereby incorporated by reference.

A heterologous gene encoding a RNAi gene of the present invention, which includes variants of the RNAi gene, includes any suitable sequence that encodes an double stranded molecule specific for a plant target RNA. Preferably, the heterologous gene is provided within an expression vector such that transformation with the vector results in expression of the double stranded RNA molecule; suitable vectors are described below.

In yet other embodiments of the present invention, a transgenic plant comprises a heterologous gene encoding a RNAi gene of the present invention operably linked to an inducible promoter, and is grown either in the presence of the an inducing agent, or is grown and then exposed to an inducing agent. In still other embodiments of the present invention, a transgenic plant comprises a heterologous gene encoding a RNAi gene of the present invention operably linked to a promoter which is either tissue specific or developmentally specific, and is grown to the point at which the tissue is developed or the developmental stage at which the developmentally-specific promoter is activated. As noted above, preferred promoters are induced by cold (including but not limited to Arabidopsis cor15a and the wheat promoter wcs120), short days (including but not limited to poplar bark storage protein promoter), or senescence (including but not limited to SAG12, an Arabidopsis senescence promoter). In another embodiment, the promoter is phloem specific (including but not limited to the soybean sucrose binding promoter and the rice promoter Ys1). Other promoters include leaf, seed and root specific promoters. In still other embodiments of the present invention, the transgenic plant comprises a RNAi gene of the present invention operably linked to constitutive promoter. In further embodiments, the transgenic plants of the present invention express at least one double) stranded RNA molecule at a level sufficient to inhibit tocopherol biosynthesis as compared to that observed in a nontransgenic plant.

The methods of the present invention are not limited to any particular plant. Indeed, a variety of plants are contemplated, including but not limited to soybean and other cultivated beans, wheat, oats, milo, sorghum, cotton, tomato, potato, tobacco, pepper, rice, corn, barley, Brassica napus (rapeseed), Cuphea species, Arabidopsis, sunflower, poplar, pineapple, banana, turf grass, and pine. Many commercial cultivars can be transformed with heterologous genes. In cases where that is not possible, non-commercial cultivars of plants can be transformed, and the trait for expression of the RNAi gene of the present invention moved to commercial cultivars by breeding techniques well-known in the art.

The methods of the present invention contemplate the use of at least one heterologous gene encoding a RNAi gene of the present invention. Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination. Such techniques are widely described in the art (See e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.).

In general, these vectors comprise a nucleic acid sequence of the invention encoding a RNAi gene of the present invention (as described above) operably linked to a promoter and other regulatory sequences (e.g., enhancers, polyadenylation signals, etc.) required for expression in a plant.

Promoters include but are not limited to constitutive promoters, tissue-, organ-, and developmentally-specific promoters, and inducible promoters. Examples of promoters include but are not limited to: constitutive promoter 35S of cauliflower mosaic virus; a wound-inducible promoter from tomato, leucine amino peptidase (“LAP,” Chao et al. (1999) Plant Physiol 120: 979-992); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267); a tetracycline-inducible promoter (U.S. Pat. No. 5,057,422); and seed-specific promoters, such as those for seed storage proteins (e.g., phaseolin, napin, oleosin, and a promoter for soybean beta conglycin (Beachy et al. (1985) EMBO J. 4: 3047-3053)). In some preferred embodiments, the promoter is a phaseolin promoter. All references cited herein are incorporated in their entirety.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (See e.g., Odell et al. (1985) Nature 313:810; Rosenberg et al. (1987) Gene, 56:125; Guerineau et al. (1991) Mol. Gen. Genet., 262:141; Proudfoot (1991) Cell, 64:671; anfacon et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells (Calais et al. (1987) Genes Develop. 1: 1183). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Calderone et al. (1984) Cell 39:499; Lassoer et al. (1991) Plant Molecular Biology 17:229), a plant translational consensus sequence (Joshi (1987) Nucleic Acids Research 15:6643), an intron (Luehrsen and Walbot (1991) Mol. Gen. Genet. 225:81), and the like, operably linked to the nucleic acid sequence encoding a polypeptide that inhibits tocopherol biosynthesis.

In preparing a construct comprising a nucleic acid sequence encoding a RNAi gene of the present invention, various DNA fragments can be manipulated, so as to provide for the DNA sequences in the desired orientation (e.g., sense or antisense) orientation. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (e.g., transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra (1982) Gene 19: 259; Bevan et al. (1983) Nature 304:184), the bar gene which confers resistance to the herbicide phosphinothricin (White et al. (1990) Nucl Acids Res. 18:1062; Spencer et al. (1990) Theor. Appl. Genet. 79:625), the hph gene which confers resistance to the antibiotic hygromycin (Blochlinger and Diggelmann (1984) Mol. Cell. Biol. 4:2929), and the dhfr gene, which confers resistance to methotrexate (Bourouis et al. (1983) EMBO J., 2:1099).

In some preferred embodiments, the vector is adapted for use in an Agrobacterium mediated transfection process (See e.g., U.S. Pat. Nos. 5,981,839; 6,051,757; 5,981,840; 5,824,877; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention are utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted polypeptide that inhibits tocopherol biosynthesis) can be expressed from these vectors as a fusion protein (e.g., coat protein fusion protein) or from its own subgenomic promoter or other promoter. Methods for the construction and use of such viruses are described in U.S. Pat. Nos. 5,846,795; 5,500,360; 5,173,410; 5,965,794; 5,977,438; and 5,866,785, all of which are incorporated herein by reference.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

Once a nucleic acid sequence encoding a polypeptide that inhibits tocopherol biosynthesis is operatively linked to an appropriate promoter and inserted into a suitable vector for the particular transformation technique utilized (e.g., one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, the vector is introduced through ballistic particle acceleration using devices (e.g., available from Agracetus, Inc., Madison, Wis. and Dupont, Inc., Wilmington, Del.). (See e.g., U.S. Pat. No. 4,945,050; and McCabe et al. (1988) Biotechnology 6:923). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Datta et al. (1990) Bio/Technology 8:736 (rice); European Patent Application EP 0 332 581 (orchardgrass and other Pooideae); Vasil et al. (1993) Biotechnology, 11: 1553 (wheat); Weeks et al. (1993) Plant Physiol., 102: 1077 (wheat); Wan et al. (1994) Plant Physiol. 104: 37 (barley); Jahne et al. (1994) Theor. Appl. Genet. 89:525 (barley); Knudsen and Muller (1991) Planta, 185:330 (barley); Umbeck et al. (1987) Bio/Technology 5: 263 (cotton); Casas et al. (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat) and Nehra et al. (1994) The Plant Journal, 5:285 (wheat).

In other embodiments, direct transformation in the plastid genome is used to introduce the vector into the plant cell (See e.g., U.S. Pat. Nos. 5,451,513; 5,545,817; 5,545,818; PCT application WO 95/16783). The basic technique for chloroplast transformation involves introducing regions of cloned plastid DNA flanking a selectable marker together with the nucleic acid encoding the RNA sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al. (1990) PNAS, 87:8526; Staub and Maliga, (1992) Plant Cell, 4:39). The presence of cloning sites between these markers allowed creation of a plastid targeting vector introduction of foreign DNA molecules (Staub and Maliga (1993) EMBO J., 12:601). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga (1993) PNAS, 90:913). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway (1985) Mol. Gen. Genet, 202:179). In still other embodiments, the vector is transferred into the plant cell by using polyethylene glycol (Krens et al. (1982) Nature, 296:72; Crossway et al. (1986) BioTechniques, 4:320); fusion of protoplasts with other entities, either minicells, cells, lysosomes or other fusible lipid-surfaced bodies (Fraley et al. (1982) Proc. Natl. Acad. Sci., USA, 79:1859); protoplast transformation (EP 0 292 435); direct gene transfer (Paszkowski et al. (1984) EMBO J., 3:2717; Hayashimoto et al. (1990) Plant Physiol. 93:857).

In still further embodiments, the vector may also be introduced into the plant cells by electroporation (Fromm, et al. (1985) Proc. Natl. Acad. Sci. USA 82:5824; Riggs et al. (1986) Proc. Natl. Acad. Sci. USA 83:5602). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In addition to direct transformation, in some embodiments, the vectors comprising a nucleic acid sequence encoding a RNAi gene of the present invention are transferred using Agrobacterium-mediated transformation (Hinchee et al. (1988) Biotechnology, 6:915; Ishida et al. (1996) Nature Biotechnology 14:745). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (e.g., nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237: 1176). Species that are susceptible to infection by Agrobacterium may be transformed in vitro. Alternatively, plants may be transformed in vivo, such as by transformation of a whole plant by Agrobacteria infiltration of adult plants, as in a “floral dip” method (Bechtold N, Ellis J, Pelletier G (1993) Cr. Acad. Sci. III-Vie 316: 1194-1199).

After selecting for transformed plant material that can express the heterologous gene encoding a RNAi gene of the present invention, whole plants are regenerated. Plant regeneration from cultured protoplasts is described in Evans et al. (1983) Handbook of Plant Cell Cultures, Vol. 1: (MacMillan Publishing Co. New York); and Vasil I. R. (ed.), Cell Culture and Somatic Cell Genetics of Plants, Acad. Press, Orlando, Vol. I (1984), and Vol. III (1986). It is known that many plants can be regenerated from cultured cells or tissues, including but not limited to all major species of crop plants, Arabidopsis, sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

Transgenic lines are established from transgenic plants by tissue culture propagation. The presence of nucleic acid sequences encoding a RNAi gene of the present invention (including mutants or variants thereof) may be transferred to related varieties by traditional plant breeding techniques.

These transgenic lines are then utilized for generation of biofuels from defective tocopherol biosynthetic plants.

A number of promoters that are active in plant cells have been described in the literature. These include the nopaline synthase (NOS) promoter as described in Ebert et al., Proc. Natl. Acad. Sci. USA, 84: 5745-5749 (1987), hereby incorporated by reference, the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Mol. Biol., 9: 315-324 (1987)) and the CaMV 35S promoter (Odell et al., Nature, 313: 810-812 (1985)), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. USA, 84: 6624-6628 (1987)), the sucrose synthase promoter (Yang et al., Proc. Natl. Acad. Sci. USA, 87: 4144-4148 (1990)), the R gene complex promoter (Chandler et al., The Plant Cell, 1: 1175-1183 (1989)), all of which are incorporated by reference, and the chlorophyll a/b binding protein gene promote. These promoters have been used to create DNA constructs that have been expressed in plants; see, e.g., WO 84/02913, hereby incorporated by reference. The CaMV 35S promoters are preferred for use in plants. Promoters known or found to cause transcription of DNA in plant cells can be used in the present invention.

For this purpose, one may choose from a number of promoters for genes with tissue- or cell-specific or enhanced expression. Examples of such promoters reported in the literature include the chloroplast glutamine synthetase GS2 promoter from pea (Edwards et al., Proc. Natl. Acad. Sci. USA, 87: 3459-3463 (1990)), the chloroplast fructose-1,6-biphosphatase (FBPase) promoter from wheat (Lloyd et al., Mol. Gen. Genet., 225: 209-216 (1991)), the nuclear photosynthetic ST-LS1 promoter from potato (Stockhaus et al., EMBO J., 8: 2445-2451 (1989)), the serine/threonine kinase (PAL) promoter and the glucoamylase (CHS) promoter from Arabidopsis thaliana. Also, reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol., 35: 773-778 (1994)), the promoter for the Cab-1 gene from wheat (Fejes et al., Plant Mol. Biol., 15: 921-932 (1990)), the promoter for the CAB-1 gene from spinach (Lubberstedt et al., Plant Physiol., 104: 997-1006 (1994)), the promoter for the cab1R gene from rice (Luan et al., Plant Cell., 4: 971-981 (1992)), the pyruvate, orthophosphate dikinase (PPDK) promoter from corn (Matsuoka et al., Proc. Natl. Acad. Sci. USA, 90: 9586-9590 (1993)), the promoter for the tobacco Lhcb1*2 gene (Cerdan et al., Plant Mol. Biol., 33: 245-255 (1997)), the Arabidopsis thaliana SUC2 sucrose-H+ symporter promoter (Truernit et al., Planta., 196: 564-570 (1995)) and the promoter for the thylakoid membrane proteins from spinach (psaD, psaF, psaE, PC, FNR, atpC, atpD, cab, rbcS), all of which are hereby incorporated by reference. Other promoters for the chlorophyll a/b-binding proteins may also be utilized in the present invention, such as the promoters for LhcB gene and PsbP gene from white mustard (Sinapis alba) as described in Kretsch et al., Plant Mol. Biol., 28:219-229 (1995) and hereby incorporated by reference.

Other promoters can also be used to express a polypeptide in specific tissues, such as seeds or fruits. Indeed, in a preferred embodiment, the promoter used is a seed specific promoter. Examples of such promoters include the 5′ regulatory regions from such genes as napin (Kridl et al., Seed Sci. Res., 1: 209:219 (1991)), phaseolin (Bustos et al., Plant Cell, 1: 839-853 (1989)), soybean trypsin inhibitor (Riggs et al., Plant Cell, 1: 609-621 (1989)), ACP (Baerson et al., Plant Mol. Biol., 22: 255-267 (1993)), stearoyl-ACP desaturase (Slocombe et al., Plant Physiol., 104:167-176 (1994)), soybean alpha' subunit of beta-conglycinin (soy7s) (Chen et al., Proc. Natl. Acad. Sci. USA, 83: 8560-8564 (1986)), and oleosin (see, for example, Hong et al., Plant Mol. Biol., 34: 549-555 (1997)), all of which are incorporated by reference. Further examples include the promoter for beta-conglycinin (Chen et al., Dev. Genet., 10: 112-122 (1989)). Also included are the zeins, which are a group of storage proteins found in corn endosperm. Genomic clones for zein genes have been isolated (Pedersen et al., Cell, 29: 1015-1026 (1982); and Russell et al., Transgenic Res., 6: 157-168), all of which are hereby incorporated by reference, and the promoters from these clones, including the 15 kD, 16 kD, 19 kD, 22 kD, 27 kD and genes, could also be used. Other promoters known to function, for example, in corn include the promoters for the following genes: waxy, brittle, shrunken 2, branching enzymes I and II, starch synthases, debranching enzymes, oleosins, glutelins and sucrose synthases. A particularly preferred promoter for corn endosperm expression is the promoter for the glutelin gene from rice, more particularly the Osgt-1 promoter as described in Zheng et al., Mol. Cell Biol., 13: 5829-5842 (1993) and hereby incorporated by reference. Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins and the glutenins. Examples of such promoters in rice include those promoters for the ADPGPP subunits, the granule bound and other starch synthase, the branching enzymes, the debranching enzymes, sucrose synthases and the glutelins. A particularly preferred promoter is the promoter for rice glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins. A preferred promoter for expression in the seed is a napin promoter. Another preferred promoter for expression is an Arcelin 5 promoter. Root specific promoters may also be used. An example of such a promoter is the promoter for the acid chitinase gene as described in Samac et al., Plant Mol. Biol., 25: 587-596 (1994), hereby incorporated by reference. Expression in root tissue could also be accomplished by utilizing the root specific subdomains of the CaMV35S promoter that have been identified as described in Lam et al., Proc. Natl. Acad. Sci. USA, 86: 7890-7894 (1989), hereby incorporated by reference. Other root cell specific promoters include those reported by Conkling et al., Plant Physiol., 93: 1203-1211 (1990), hereby incorporated by reference. Other preferred promoters include 7-alpha' as described in Beachy et al., EMBO J., 4: 3047 (1985); Schuler et al., Nucleic Acid Res., 10: 8225-8244 (1982); USP 88 and enhanced USP 88 (U.S. Patent Ser. No. 60/377,236, filed May 3, 2002, incorporated herein by reference); and 7S-alpha, (U.S. patent application Ser. No. 10/235,618). Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436, all of which are incorporated by reference. In addition, a tissue specific enhancer may be used as described by Fromm et al., The Plant Cell, 1: 977-984 (1989), hereby incorporated by reference. Still other preferred promoters include the Arabidopsis cor15a, the inducible wheat promoter wcs120, the poplar bark storage protein gene promoter, the Arabidopsis senescence promoter SAG12, the maize cysteine protease SEE promoter, the soybean sucrose binding promoter and the rice promoter Ys1.

### EXAMPLES

### Example 1

### Growth Condition and HL and Low Temperature Treatment

Seeds were stratified for four to seven days at 4° C., planted in a vermiculate and soil mixture, fertilized with 1× Hoagland solution and grown in a chamber under permissive conditions; 12 h, 120 μmol photon/m²/s light at 22° C./12 hr darkness at 18° C. with 70% relative humidity. Plants were watered every other day and with 0.5× Hoagland solution once a week. For HL treatments, four week old plants were transferred in the middle of the light cycle to 1800 μmmol photon/m²/s light/16 hr light/8 hr darkness at 22° C. For low temperature treatments, three to four week old plants were transferred at the beginning of light cycle to 12 hr, μmol photon/m²/s light/12 hr darkness at 7.5° C. (±<3° C.).

### Example 2

### Tocopherol, Anthocyanin, Chlorophyll and Carotenoid Analysis

Leaf samples (12-15 mg) were harvested directly into liquid nitrogen at the end of the light cycle and lipids extracted in the presence of 0.01% (w/v) butylated hydroxytoluene (BHT) using tocol as an internal standard as previously described (Collakova and DellaPenna, 2001). After phase separation, the aqueous phase was transferred to a new tube, acidified by adding an equal volume of 1N HCl and anthocyanin content measured spectrophotometrically at 520 nm as previously described (Merzlyak and Chivunova, 2000, J. Photochem. Photobiol. B 55:155-163, incorporated herein in its entirety). The lipid phast was used for reverse-phase HPLC analysis to adentify and quantify each tocopherol, chlorophyll and carotenoid species as previously described (Collakova nad DellaPenna, 2001; Tian and DellaPenna, 2001, Plant Mol. Biol. 47:379-388, incorporated herein in its entirety).

### Example 3

### Lipid Peroxidase Analysis

Lipid peroxidase content was measured using the ferrous oxidation-xylenol orange (FOX) assay as previously described (DeLong et al, 2002, J. Agr. Food Chem. 50:248-254; Sattler et al., 2004, incorporated herein in their entireties) with the following modifications. Leaf samples (25-30 mg) harvested at the end of light cycle were immediately extracted with 200 μl methanol containing 0.01% (w/v) BHT, 200 μl of dichloromethane, and 50 μl of 150 μM acetic acid using three 3 mm glass beads and a commercial paint shaker. After shaking for 4 min, 100 μl of water and 100 μl of dichloromethane were added for phase separation. Half of the organic phase was incubated with an equal volume of 50 mM triphenyl phosphine in methanol for 30 min to reduce lipid peroxides and half was incubated with an equal volume of methanol for 30 min. The triphenyl phosphine treated and untreated samples (100 μl) were incubated with 900 μl of FOX reagent (90% (v/v) methanol, 4 mM BHT, 25 mM sulfuric acid, 250 μM ferrous ammonium sulfate, 100 μM xylenol orange) at room temperature for exactly 20 min and A₅₆₀ was measured. Lipid peroxidase content wa calculated basedo n a standard curve of hydrogen peroxide as previously described (DeLong et al., 2002).

### Example 3

### Chlorophyll Fluorescence Measurements

In vivo chlorophyll a fluorescence was measured in the middle of the light cycle using a pulse amplitude modulation (PAM) fluorometer FMS2 (PP Systems, Haverhill, Mass.). Attached leaves were dark adapted for at least 15 min prior to measurements and fluorescence parameters were determined according to Maxwell and Johnson (2000, J. Exp. Bot. 51:659-668, incorporated herein in its entirety). Electron transport rate (ETR) was calculated as PFDa×ΦPSII×0.5, where PDFa is actinic light (100 μmol photon/m²/s) measured by a LI-250 Light Meter (LI-COR Inc., NE). ΦPSII=(F′m-Ft)/F′m, the efficiency of PSII photochemistry (F'm, maximum fluorescence in the light, Ft, steady state fluorescence in the light).

### Example 4

### Carbohydrate Analyses

Soluble sugar (e.g., glucose, fructose, sucrose) and starch levels of leaves were quantified as previously described (Jones et al., 1977, Plant Physiol. 60:379-383; Lin et al., 1988, Plant Physiol., 86:1131-1135, incorporated herein in their entireties) with minor modifications. Unshaded leaf tissue (<50 mg) was harvested, immediately frozen in liquid nitrogen and extracted twice with 700 μl of 80% ethanol at 80° C. The ethanol extract was evaporated and redissolved in 200 μl of distilled water (Jones et al., 1977). For starch analysis, the extracted leaf residue was ground in 200 μl of 0.2N KOH and boiled at 95° C. for 45 min. After cooling, the sample was neutralized to pH 5 with 50 μl of 1N acetic acid, centrifuged and 50 μl of supernatant mixed with 492.5 μl of 0.2M sodium acetate (pH 4.8), 150 μl of water, 4 μl of α-amylase (4 units) and 3.5 μl of amyloglucosidase (2 units) and incubated at 37° C. overnight. Glucose, fructose and sucrose levels in the soluble sugar extract and the glucose level of the digested starch extract were determined enzymatically (Jones et al., 1997).

### Example 5

### ¹⁴CO₂ Labeling

For phloem exudation experiments, approximately 20 mature leaves (7^{th} to 9^{th} oldest) were detached in the middle of the day, 0.5 cm of petiole was re-cut under water, the petiole of each leaf was submerged in water and placed in a tightly sealed 10 liter glass chamber. ¹⁴CO₂ was generated in the chamber by adding 3 ml of 0.25N H₂SO₄ to 0.1 mCi (7 μmol) NaH¹⁴CO₃ and unlabeled 93 μmol NaHCO₃ to give a carbon dioxide concentration of 522 ppm. After labeling for 30 min at 120 μmol photon/m²/s light, the petiole of each leaf was submerged in 0.45 ml of 20 mM EDTA (pH 7.0) and kept in the dark with high humidity to induce phloem exudation. Aforementioned procedures were performed at 7.5° C. for low temperature treated leaf samples. Exudation of radiolabel into the EDTA solution was then periodically measured over the course of 10 hr by liquid scintillation counting (Tri-Carb 2800TR; PerkinElmer, Wellesley, Mass.). After 10 hr of exudation, radiolabel remaining in the leaves was determined by liquid scintillation counting. Total radiolabel fixed per leaf was calculated by adding total radiolabel exuded and remaining in each leaf.

For analyzing translocation of radiolabeled photoassimilates in whole plants, plants were grown on ½ MS plates under permissive conditions for 3 weeks and transferred to low temperature conditions for seven days with lids partially afar to supply atmospheric CO₂. Whole plants were labeled at 7.5° C. for 40 min as described above, placed in darkness at 7.5° C. and high humidity for 2 hr to allow for translocation. Roots were excised from leaves and both exposed to a phosphor screen to visualize the location of radioactivity (Storm, GE Healthcare, UK).

Carbohydrate analysis of phloem exudates was performed by high pH anion exchange chromatography (HPAEC). Excised leaves were treated as described for phloem exudation experiments except at the end of the initial 2 hr exudation period the petiole of each leaf was transferred to 0.45 ml water for 4 hr to collect exudates for analysis. The water exudates were dried under vacuum, dissolved in 50 μl water and 20 μl of the sample was mixed with 5 μl of standards (25 nmol glucose, 50 nmol fructose, 125 nmol sucrose and 100 nmol raffinose) and injected onto the HPAEC. The mixtures were separated on a CarboPac PA-10 column (DIONEX, Sunnyvale, Calif.) using a 30 min linear gradient of 20 to 140 mM NaOH with a flow rate of 1 ml/min. One ml fractions were collected and radioactivity was determined by liquod scintillation counting, while sugar standards were detected by pulsed amperometric detection.

### Example 6

### Fluorescence and Transmission Electron Microscopy

Leaves were prepared for aniline blue fluorescence microscopy (n=2 leaves/plant, 4-6 plants/sample time; Martin, 1959, Stain Tech. 34:125-128, incorporated herein in its entirety) and transmission electron microscopy (TEM; n=1 leaf/plant, 2-3 plants/sample time; Sage and Williams, 1995, Sex. Plant Reprod. 8:257-265, incorporated herein in its entirety) at the same sampling times as above for export studies. The presence or absence of callose was determined using immunolocalization at the level of the TEM as described by Lam et al. (2001, Plant Cell 13:2499-2512, incorporated herein in its entirety) with monoclonal antibodies to β-1,3 glucan (BioSupplies, Australia). Primary and secondary (anti-mouse IgG gold conjugate 18 nm, Jackson Immunoresearch, West Grove, Pa.) antibody dilutions were 1:100 and 1:20, respectively. Incubation time in the primary and secondary antibodies were 2 and 1 hr, respectively. Controls were run by omitting the primary antibody. Images were captured on the Leica MZ 16F fluorescnec microscope (Wetzlar, Germany) and the Phillips 201 TEM equipped with an Advantage HR Camera System (Advanced Microscopy Techniques Corp., Danvers, Mass.).

### Example 7

### Tocopherol Biosynthetic (vte1) Mutants

Vte1, vte2 and vte2-1 were previously isolated and characterized ethyl mthanesulfonate mutants in the Columbia (Col) ecotype and are deficient in the tocopherol cyclase and HPT enzymes, respectively (Sattler et al., 2003, FIG. 1). Vte2-2 and vte4-3 are T-DNA insertion mutants in the Wassilewskija (Ws) ecotype in genes encoding HPT and γ-tocopherol methyltransferase (γ-TMT), respectively. Leaves of all mutants, vte2-1, vte2-2, vte1-1, vte1-2 and vte4-3, lack α-tocopherol, the major tocopherol in wild type Arabidopsis leaves (FIG. 1, Table 1, Sattler et al., 2003). Vte2-1 and vte2-2 lack all tocopherols and pathway intermediates. Vte1-1 and vte1-2 lack all tocopherols but accumulate the biosynthetic pathway intermediate DMPBQ at a level comparable to α-tocopherol in Col. The vte4-3 mutant accumulates γ-tocopherol at an equivalent or slightly higher level than α-tocopherol in Ws. Three to five week old plants of all mutant genotypes grown under permissive conditions (12 hr at 120 μmol/m²/s light at 22° C./12 hr darkness at 18° C.) were virtually identical to their respective wild type backgrounds, consistent with previous reports that mutations disrupting tocopherol synthesis have little impact on the normal growth of mature plants (Porfirova et al., 2002; Bergmuller et al., 2003; Sattler et al., 2003 and 2004).

### Example 8

### Biofuel Production

Tocopherol biosynthesis mutants as described in Example 7 can be used to make biofuels, such as ethanol. Equally useful and applicable would be crop plants wherein the combination of defective tocopherol biosynthesis and altered carbon allocation of soluble sugars and starch to leaves instead of roots or other plant structures is demonstrated. As an example, one may design inducible promoters that regulate the expression of VTE genes, e.g., VTE1 and VTE2 in Arabidopsis as well as the homologous tocopherol cyclase (VTE1 homologs) and homogentisate prenyltransferase (VTE2) genes in suitable crop plants, which include but are in no way limited to maize (corn), cotton, sunflower, soybean, rice, cider trees and tubers including potatoes as described in United States Patent Application Number 2005/0203047, hereby incorporated by reference. As exemplified in Tables 5 and 6, the VTE genes in such crops share significant homology with the homologous suite of genes in Arabidopsis. Thus, the design of inducible promoters appropriate for promoting levels of expression in VTE1- and VTE2-homologous genes is routinely performed when armed with the nucleotide sequence for the gene, as available from the National Center for Biotechnology Information. Subsequently, promoter binding sites may be determined using prediction software such as the Promoter Prediction program developed by Lawrence Berkeley National Laboratory.

For example, mutants plants as described herein are milled and ground into a powder. The resultant powder is enzymatically liquefied (e.g., by the addition of a amylase or other such enzyme useful in liquefying starch). Following liquefaction, the liquefied starch is converted to a fermentable sugar with the addition of a secondary enzyme (e.g., gluco-amylase). This process is typically known as saccharification. The soluble sugars and converted starch are subsequently fermented by the addition of yeast or bacteria, which serves to ferment the sugars to ethanol and carbon dioxide. The Ethanol is the distilled, dehydrated, and denatured, thereby creating ethanol that is useful as a biofuel and unsafe for human consumption. Those skilled in the art will recognize permutations and modifications of the above-mentioned procedures that will result in the same biofuel production.

### Results

HL stress results in excessive excitation of chlorophyll and consequently generates reactive oxygen species (ROS), which in turn attack various biochemical targets in the cell including PUFA enriched photosynthetic membranes. Tocopherols are most abundant in these phoptosynthetic membranes (Bucke, 1968; Lichtenthaler et al., 1981; Soll et al., 1985) and leaf tocopherol levels increase up to 18-fold during HL stress in Arabidopsis (Collakova and DellaPenna, 2003). Therefore, it has been presumed that the elimination of tocopherols from photosynthetic membranes would have dramatic impacts on plant survival during HL stress. To test this hypothesis, Col, vte2-1, vte1-1 and vte1-2 were grown for four weeks under permissive conditions and then subjected to two levels of HL stress, 1000 and 1800 μmol/m²/s 16 hr light/8 hr darkness at 22° C. (hereafter referred to as HL1000 and HL1800, respectively). HL1000 did not result in differential visible or biochemical phenotypes between vte2-1 and Col. When Col, vte2-1, vte1-1 and vte1-2 were subjected to HL1800, which approaches the intensity of full sunlight, the led to bleaching of some mature leaves in all genotypes. Vte2-1 had a slight tendency toward more bleached leaves than Col but this was not reproducible or significant, while vte-1 and vte1-2 reproducibly had as many or more bleached mature leaves than Col or vte2-1. vte2-2 and vte4-3 subjected to HL1800 responded similarly to Ws, the corresponding wild type.

To Access changes in photosynthetic pigment an tocopherol levels in response to HL stress, the 7^{th} to 9^{th} oldest leaves were harvested before and after 4 days of HL 1800 for HPLC analysis. Before HL1800, all levels were similar between genotypes except for the absence of tocopherols in vte genotypes (Table 2). After 4 days of HL1800, the total tocopherol level in Col increased approximately 10-fold, while no tocopherol was detected in all vte mutants. The vte mutants generally had lower total and individual chlorophyll levels than Col, but these differences were not significan in all cases after 4 days of HL1800 (Table 2). Total carotenoids were consistently and significantly lower than Col in vte1-1 and vte1-2, but not always in vte2-1 after 4 days of HL1800. Neoxanthin and violaxanthin were significantly lower in all vte mutants, while lutein was significantly lower only in vte1-1 and vte1-2. Interestingly, zeaxanthin was 70% higher than Col in vte2-1 but unchanged relative to Col in both vte1 alleles. In comparing the impact of HL on photosynthetic pigments between vte 1 and vte 2 genotypes, three significant differences stand out; both vte1 alleles have higher violaxanthin levels, lower zeaxanthin levels and a lower de-epoxidation state (A+Z/V+A+Z) than vte2-1.

In vivo chlorophyll a fluorescence was analyzed to access PSII function during HL stress. Typically, when plants are under oxidative stress, PSII is inactivated due to enhanced turnover of the D1 protein, a process termed photo inhibition, and maximum photosynthetic efficiency (Fv/Fm) decreases (Maxwell and Johnson, 2000). Four week old Col, vte2-1, vte1-1 and vte1-2 plants grown under permissive conditions had identical Fv/Fm values of between 0.8 and 0.85, typical values for healthy leaves (Maxwell and Johnson, 2000, Table 2). After 24 hr of HL1800 (8 hr HL1800, 8 hr darkness, 8 hr HL1800), a few vte2-1 leaves showed a dramatic reduction in Fv/Fm (<0.5), but the majority had values similar to Col and the average Fv/Fm of vte2-1 was not significantly different from Col (Table 2). In contrast, vte1-1 and vte1-2 both had more leaves with Fv/Fm <0.5 and average Fv/Fm values that were significantly lower than Col (Table 2).

These combined results indicated that the elimintation of tocopherols in vte2 has little impact on the response of the photosynthetic apparatus to HL stress in comparison to Col, with the exception of altered carotenoid compositions. Though the vte2 and vte1 genetoypes are identical with regard to their tocopherol deficiencies, vte1 alleles are more susceptible to HL1800 than vte2. As the primary biochemical difference between these two genotypes is that vte1 mutants accumulate the redox active intermediate DMPBQ while vte2 mutants do not, it is contemplated that the presence of DMPBQ in vte1 may have negative impacts on HL stress tolerance in Arabidopsis.

Wild type and tocopherol deficient mutants Col and vte2-1 were grown under permissive conditions for three to five weeks and subjected to abiotic stress treatments other than HL, including salinity (100, 150 and 200 nM NaCl), drought and various low temperature treatments. Like HL stress, the salinity and drought stress conditions used did not result in obvious phenotypic differences between vte2-1 and Col. However, when plants were transferred from permissive conditions to non-freezing low temperature conditions, the vte2 genotypes became readily distinguishable from wild type. Both vte2-1 and vte2-2 grew more slowly than their respective wild types, Col and Ws, and their mature leaves changed color to purple. These phenotypic differences were consistently observed in conditions ranging from 3° C. to 12° C. and light intensities from 15 to 200 mmol/m²/s. Differences were most obvious and consistent when plants were transferred from permissive conditions to 7.5° C., 12 hr 75 μmol/m²/s light/12 hr darkness and this low temperature regimen (hereafter termed 7.5° C.-treated) was used for all subsequent experiments.

Following transfer to 7.5° C., vte2-1 and Col did not differ in time to bolting (53±4 and 51±3 days, respectively, after transfer to 7.5° C.) or number of leaves produced at the start of bolting (32±3 and 31±2 leaves, respectively), indicating that the process of vernalization was not affected by the lack of tocopherols. However, after prolonged growth at 7.5° C. vte2-1 siliques were shorter, produced significantly fewer seeds per silique and per plant compared to Col, and 35% of the seeds in vte2-1 siliques were aborted compared to <1% in Col siliques (Table 3). These results indicate tocopherols play a role in low temperature adaptation in Arabidopsis.

Subjecting vte1-1 to 7.5° C. treatment resulted in a phenotype intermediate between vte2-1 and Col in terms of overall growth, mature leaf color, silique size, number of seeds per silique, percentage of aborted seeds and seed yield per plants (Table 3). These phenotypes in 7.5° C.-treated vte4-3 were virtually indistinguishable from Ws. These results indicate that during low temperature adaptation in Arabidopsis the quinol biosynthetic intermediate DMPBQ partially compensates for the lack of tocopherols in vte 1-1 while the γ-tocopherol accumulated in vte4-3 leaves can functionally replace-α-tocopherol in this regard.

Vte2-1 and Col were subject to detailed comparative biochemical analysis. Plants grown for four weeks at permissive conditions were transferred to 7.5° C. conditions and the 7^{th} and 9^{th} oldest fully expanded rosette leaves were harvested at various time points for analysis. The tocopherol content in Col started to increase after 3 days of 7.5° C. treatment reaching levels 5-fold high than initial levels by 28 days of treatment, while vte2-1 lacked tocophereols at all time points, consistent with the nature of this mutation (FIG. 2A).

The purple color of mature leaves of 7.5° C.-treated vte2 mutants suggested the accumulation of anthocyanins. Indeed, both Col and vte2-1 accumulated low but detectably elevated levels of anthocyanins after 7 days at 7.5° C. (0.12 nmol/mg FW), after which time anthocyanins in Col decreased to background levels while the levels in vte2-1 continued to increase up to 2 nmol/mg FW by 28 days (FIG. 2D). Because anthocyanin accumulation is often associated with plant responses to stress (Leyva et al., 1995, Plant Physiol. 108:39-46; Chalker-Scott, 1999, Photochem. Photobiol. 70:1-9, incorporated herein in their entireties) and tocopherols are well-characterized lipid soluble antioxidants in animals (Ham and Liebler, 1995 and 1997), it is contemplated that elevated lipid peroxidation might be occurring in vte2-1 during low temperature treatment. However, the lipid peroxide levels of vte2-1 and Col analyzed by the FOX assay were found to be similar and near background levels at all time points (FIG. 2B), indicating that the observed phenotypic differences between 7.5° C.-treated vte2-1 and Col are not associated with a detectable increase in lipid peroxidation.

Given the reported localization of tocopherols and tocopherol biosynthetic enzymes to plastids (Bucke, 1968; Soll et al., 1980, 1985 and 1987; Lichtenthaler et al., 1981), tocopherol deficiency might affect the components and function of the photosynthetic apparatus during 7.5° C. treatment. Under permissive growth conditions, the levels of individual and total photosynthetic pigments (chlorophylls and carotenoids) were nearly identical in vte2-1 and Col (FIGS. 2C and E, Table 4). The chlorophyll and carotenoid conent of both vte2-1 and Col changed in parallel during the first two weeks of 7.5° C. treatment and became significantly different only at 28 days (FIGS. 2C and E, Table 4). Zeaxanthin, a xanthophyll cycle carotenoid that accumulated under HL stress (Table 2), was not detectable at any time point in 7.5° C.-treated Col and vte2-1 (Table 4), suggesting that the plants were not experiencing photooxidative stress under the low temperature conditions used.

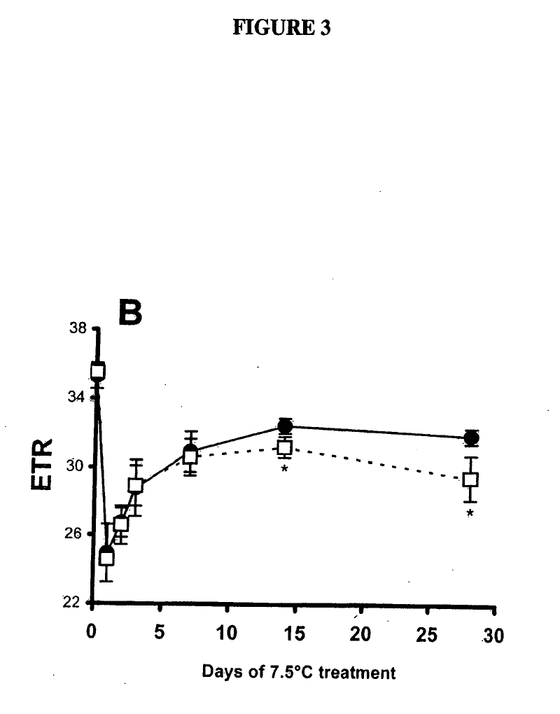

To assess the response of the photosynthetic apparatus to 7.5° C. conditions, changes in photosynthetic parameters were analyzed. Fv/Fm was unchanged in both Col and vte2-1 at any time point, indicating that photoinhibition is not occurring in either genotype during permissive or 7.5° C. conditions. The electron transport rate (ETR) of Col and vte2-1 was also identical under permissive growth conditions, indicating that tocopherol deficiency also does not affect the rate of linear photosynthetic electron transport in the absence of stress. During the first 7 days of 7.5° C. treatment, the ETR responded identically in Col and vte2-1: ETR decreased sharply during the first day followed by a gradual recovery by 7 days. However, at 14 days the vte2-1 ETR was significantly lower than Col and declined further by 28 days, while the ETR of Col remained stable from day 14 and onward (FIG. 3).

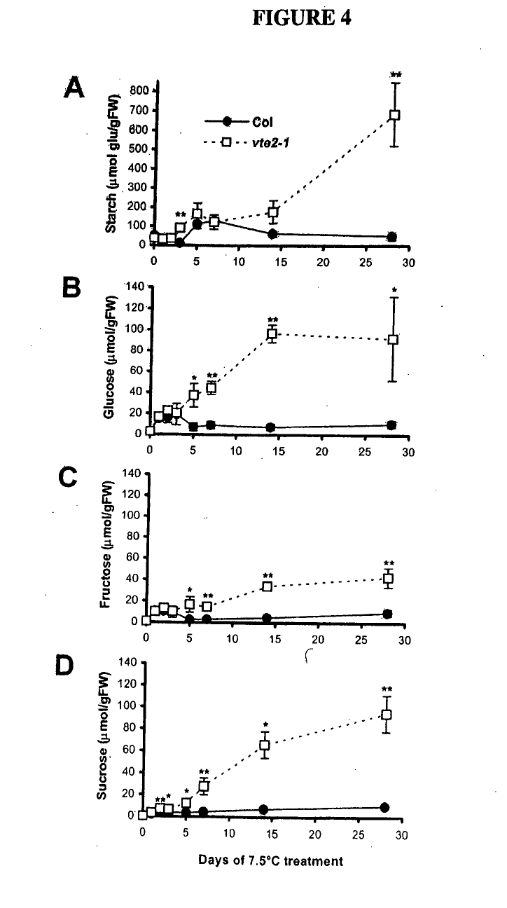

It is contemplated that the reduced ETR in vte2-1 after 14 days could result from feedback inhibition of photosynthesis due to the accumulation of downstream carbon metabolites (Goldschmidt and Huber, 1992, Plant Physiol. 99:1443-1448; Koch, 1996, Ann. Rev. Plant Physiol. Plant Mol. Biol. 47:509-540; Paul and Foyer, 2001, J. Exp. Bot. 52:1383-1400; Paul and Peliny, 2003, J. Exp. Bot. 54:539-547, incorporated herein in their entireties). To assess this possibility, starch, glucose, fructose and sucrose contents were analyzed during the time course of 7.5° C. treatment. Starch represents the main plastidic carbohydrate storage pool, sucrose and fructose are cytosolic pools, while glucose is present in both subcellular compartments. Col and vte2-1 had identical carbohydrate contents at the end of the light period under permissive growth conditions. During the first 7 days of 7.5° C. treatment starch content increased similarly in both Col and vte2-1 to approximately 120 mmol glucose equivalents/g FW. After 7 days vte2-1 starch content steadily increased to 680 μmol glucose equivalents/g FW while Col starch levels decreased to near initial levels (FIG. 4A). Likewise, the glucose, fructose and sucrose content of Col and vte2-1 increased similarly during the first 3 days of low temperature treatment (FIGS. 4B, C and D). After 3 days, Col soluble sugar levels decreased, while vte2-1 continued to rise reaching 35, 43 and 255 times the initial levels of glucose, fructose and sucrose, respectively, after 28 days of low temperature treatment. The timing of the increase and accumulation of carbohydrates in vte2-1 is consistent with this being the root cause of the reduction in ETR observed after 14 days at 7.5° C. (FIG. 3).

To further investigate any differences in carbohydrate accumulation between vte2-1 and Col during the initial 5 days of low temperature treatment, diurnal changes in carbohydrate content were analyzed one hour before the end of the light and dark cycles. During the 25 hr prior to low temperature treatment (FIG. 5; −25 h, −13 h and −1 h, with 0 h being the transfer of plants to low temperature at the start of the light cycle), starch, glucose, fructose and sucrose content were identical in vte2-1 and Col. These data indicate the lack of tocopherols does not have a significant impact on carbohydrate metabolism under permissive growth conditions. Following transfer to low temperature the soluble sugar content increased similarly in vte2-1 and Col for the first two diurnal cycles with significant differences first being observed between genotypes at the end of the third low temperature light period (59 hr in FIGS. 5B, C and D). In contrast, starch levels did not become significantly different between genotypes until 14 days of low temperature treatment (FIGS. 4A and 5A). The differential elevation of soluble sugars prior to starch accumulation in vte2-1 indicates that the increase in cytosolic soluble sugars precedes starch accumulation in the chloroplast and that soluble sugars are not being efficiently metabolized or mobilized in 7.5° C.-treated vte2-1.

Mature (7^{th} to 9^{th} oldest) and young (13^{th} to 16^{th} oldest) leaves of vte2-1 and vte1-1 mutants showed obvious visible differences in their responses to low temperature; young leaves of vte2-1 and vte1-1 did not change their color to purple even after two months of 7.5° C. treatment. Mature and young leaves of Col, vte2-1 and vte1-1 were harvested after 28 days of low temperature treatment for a series of comparative biochemical analyses. Consistent with visual observations, mature leaves of vte 1-1 had an anthocyanin content 10% that of vte2-1 but still higher than Col, while young vte2-1 and vte1-1 leaves accumulated much less anthocyanins compared to their respective mature leaves (FIG. 6A). Fv/Fm was above 0.8 in all cases, indicating that photoinhibition was not occurring in either young or mature leaves of any genotype (FIG. 6B). The ETR of mature vte2-1 leaves was reduced to 70% of mature Col leaves, while the ETR of mature vte1-1 leaves was only slightly decreased relative to mature Col leaves. However, the ETR of mature and young Col leaves and young vte2-1 and vte1-1 leaves were not significantly different (FIG. 6C). Levels of all carbohydrates in mature vte2-1 leaves were greatly elevated in comparison to Col, consistent with FIG. 4. Mature vte1-1 leaves contained intermediate levels of starch, glucose, fructose and sucrose (51, 53, 68 and 58% of vte2-1 levels, respectively) (FIGS. 6D-G). Young vte2-1 and vte1-1 leaves contained substantially reduced starch, glucose, and sucrose levels compared to their respective mature leaves. These results indicate that the initiation and development of young vte2-1 and vte1-1 leaves under 7.5° C. conditions attenuates the biochemical phenotypes observed in mature leaves of both genotypes and that the DMPBQ accumulated in vte1-1 further suppresses these biochemical phenotypes in both mature and young leaves.

The reduced seed yield (Table 3) and attenuated carbohydrate accumulation in young leaves relative to mature leaves (FIG. 6) in 7.5° C.-treated vte2-1 suggests impaired translocation of photoassimilates from mature source tissues to young sink tissues. To test this possibility ¹⁴CO₂ labeling experiments were conducted. Col and vte2-1 were grown on plates under permissive conditions for three weeks and then transferred to 7.5° C. for an additional 7 days. Whole plants were labeled with ¹⁴CO₂ at 7.5° C., transferred to high humidity in darkness at 7.5° C. for 2 hr to allow for photoassimilate transport and subsequently exposed to a phosphor screen to visualize the movement of ¹⁴C labeled photoassimilate. Immediately after labeling >99% of the ¹⁴CO₂ incorporated) was present in leaf tissue. Col and vte2-1 incorporated similar amounts of ¹⁴CO₂ into photosynthate suggesting their carbon fixation rates do not differ, consistent with the similar ETRs within the first 7 days at 7.5° C. Following the 2 hr dark period Col had translocated 13.2% of the ¹⁴C labeled photoassimilate fixed in leaves to roots, whereas only 2.7% was translocated in vte2-1. These results demonstrate that vte2-1 translocates significantly less photoassimilate from source to sink than Col after 7 days of 7.5° C.-treatment. It is contemplated that impaired photoassimilate translocation in 7.5° C.-treated vte2-1 could be due to reduced sink strength or impaired photoassimilate export from source leaves.

To address these possibilities, phloem exudation experiments were conducted.) Col and vte2-1 were grown for four weeks at permissive conditions and transferred to 7.5° C. for an additional 0, 1, 3, or 7 days. Mature (7^{th} to 9^{th} oldest) leaves were excised from plants and labeled with ¹⁴CO₂. The petioles of labeled leaves were transferred to an EDTA solution to induce phloem exudation and radioactivity in the EDTA solution was determined at various time points (King and Zeevart, 1974). Again, total ¹⁴CO₂ fixed in mature leaves were similar in all genotypes at each time point. Prior to 7.5° C. treatment, Col, vte1-1 and vte2-1 leaves exuded similar amounts of labeled photoassimilates, accounting for approximately 34% of the total ¹⁴CO₂ fixed in each genotype. During 7.5° C. treatment, the percent exudation by Col slightly decreased after 3 and 7 days (to 27 and 31% of the total ¹⁴CO₂ fixed, respectively), whereas that of vte2-1 was greatly reduced (to 11% and 4% at 3 and 7 days, respectively). Exudation in vte2-1 was significantly lower than Col during the first day of 7.5° C. treatment, which corresponds to only 6 h at 7.5° C. The vte1-1 mutant exuded 17 and 15% of the total ¹⁴CO₂ fixed after 3 and 7 days at 7.5° C., respectively, levels intermediate between Col and vte2-1 (FIG. 7B). In apoplastic loaders like Arabidopsis sucrose is almost the exclusive translocated photoassimilate (Vanbel, 1993, Ann. Rev. Plant Physiol. Plant Mol Biol. 44:253-281, incorporated herein in its entirety). To assess the chemical nature of the labeled compounds exuded from Col and vte2-1, phloem exudates were collected and separated by anion-exchange chromatography together with sugar standards. As shown in FIG. 7A, approximately 85% of the label in Col and vte2-1 exudates co-migrated with the sucrose standard and 10% with glucose/fructose standards. The high proportion of sucrose indicates that the label collected is almost entirely from phloem exudate rather than sugars from the cytosol of damaged cells. Overall, the results obtained from ¹⁴CO₂ labeling experiments indicate that tocopherol deficiency in both vte1-1 and vte2-1 results in dramatically reduced capacity of photoassimilate export from source leaves in response to 7.5° C. treatment. The rapidity of the reduction in photoassimilate export in 7.5° C.-treated vte2-1 strongly suggests that impairment of photoassimilate export is the root cause of the sugar accumulation phenotype observed in mature leaves of 7.5° C.-treated tocopherol-deficient mutants.

Previously, callose was reported to accumulate at the bundle sheath/vascular parenchyma interface of the maize sxd1 mutant and in vascular tissue of potato vte1-RNAi lines, both of which are defective in tocopherol cyclase (Botha et al., 2000, Protoplasma 214:65-72; Hofius et al., 2004, herein incorporated in their entireties). To determine whether callose deposition also occurs in Col, vte2-1 and vte1-1, leaves were harvested at 0, 1, 3 and 13 days of 7.5° C. treatment and aniline blue-positive fluorescence assessed. Under permissive conditions, aniline blue-positive fluorescence was absent or sporadic and no significant differences were observed in any genotypes. Aniline blue-positive fluorescence was also not altered in Col during the entire 7.5° C. treatment period (e.g., 13 days at 7.5° C.). In contrast, aniline blue-positive fluorescence strongly increased in the vascular tissue of 7.5° C.-treated vte2-1, and to a slightly lesser extent vte1-1. In both vte2-1 and vte1-1, fluorescence initially appeared in a limited number of vascular cells in the petiole as early as 6 h after transfer to 7.5° C. conditions. The number of aniline blue fluorescing cells in the vasculature and their fluorescent intensity subsequently increased in an acropetal fashion in both vte2-1 and vte1-1 during the course of 7.5° C. treatment. Intriguingly, the induction, intensity and acropetal spread of vasculature-specific aniline blue positive fluorescence in vte2-1 at 7.5° C. was unaffected by light levels ranging from 1 to 800 μmol photon/m²/s. Aniline blue positive fluorescence was not observed in the vasculature of Col at any light level at 7.5° C. and was also absent from the vasculature of both Col and vte2-1 subjected to HL1800 at 22° C. for up to 4 days.

To confirm whether or not aniline blue-positive fluorescence was cell specific and could be attributed to callose deposition, serial sections of 0 and 14 day 7.5° C.-treated Col and vte2-1 vascular tissue were examined at the level of the transmission electron microscope (TEM). The spatial organization of cells and types of cells comprising the phloem and xylem of both Col and vte2-1 were identical to what has been previously described for Arabidopsis (Haritatos et al., 2000, Planta 211:105-111, incorporated herein in its entirety). Notably, at day 0 phloem vascular parenchyma cells of both Col and vte2-1 contained transfer cell wall ingrowths adjacent to sieve elements and companion cells. 7.5° C.-treatment of Col for 14 days did not result in obvious ultrastructural changes in any vascular cell type except for a noticeable increase in phloem parenchyma transfer cell differentiation and transfer cell wall deposition exclusively adjacent to sieve elements and companion cells in all vascular tissue although to a lesser degree in the midvein. During the same time course of 7.5° C.-treatment in vte2-1, changes in cell fine structure occurred exclusively within the phloem parenchyma transfer cells of all vascular traces. Phloem parenchyma transfer cells in 14 day-treated vte2-1 exhibited irregularly thickened cell wall depositions with ultrastructural features characteristic of callose. Large callosic-like masses that dissected the cell lumen corresponded in shape to aniline blue-positive fluorescent regions. The callosic-like wall material also formed a sheath around the cells, was deposited over transfer cell wall ingrowths and between the end walls of adjoining transfer cells including plasmodesmata. Immunolocalization using monoclonal antibodies against callose confirmed the presence of callose at each location and at plasmodesmata between the phloem parenchyma transfer cells and bundle sheath. No immunolabelling was present in controls using secondary antibody only and immunolabelling was rare to absent in all cell types of untreated Col and vte2-1 and in 14-day 7.5° C.-treated Col, including phloem parenchyma transfer cells.

Serial sections of vascular tissue from Col and vte2-1 treated at 7.5° C. for 3 and 7 days were subsequently examined at the level of the TEM to determine the spatial and temporal development of callose deposition within phloem parenchyma cells. At 3 days, phloem parenchyma transfer cell wall deposition in Col was confined to the sieve element or companion cell boundary, but in vte2-1 wall deposition was present around the entire transfer cell periphery. Cell wall deposition in 3 day-treated vte2-1 resulted in abnormally thickened and irregular shaped ingrowths with callose-like depositions adjacent to sieve elements and companion cells that grew increasingly prominent by day 7. In 3-day 7.5° C.-treated vte2-1 positive immunolocalization with monoclonal antibodies to callose was present exclusively at the phloem parenchyma transfer cell wall-sieve element boundary and included phloem parenchyma transfer cell-sieve element plasmodesmatal connections. In contrast to 14 day 7.5° C.-treated vte2-1, plasmodesmata between bundle sheath and phloem vascular parenchyma cells in 3 day 7.5° C.-treated vte2-1 were continuous and immunonegative for callose.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A plant deficient in tocopherol biosynthesis wherein said plant, in the presence of low, non-freezing temperatures, alters carbon allocation such that increased levels of soluble sugars and starch are found in leaf structures relative to levels found in wild type plants under the same low, non-freezing temperature conditions.

2. A plant of claim 1, wherein said plant is a species of Arabidopsis.

3. A plant of claim 1, wherein said plant is a crop plant.

4. A plant of claim 3, wherein said crop plant is maize.

5. A method for generating biofuels, comprising:
a) providing a plant of claim 1,
b) subjecting said plant to low, non-freezing temperatures such that said plant demonstrates altered carbon allocation to leaves relative to a wild type plant, and
c) subjecting said plant that demonstrates said altered carbon allocation to processes for generating a biofuel usable as an energy source.

6. The method of claim 5, wherein said biofuel usable as an energy source is ethanol.

7. A transgenic plant comprising heterologous nucleic acid sequences encoding a double stranded RNA sequence, wherein said double stranded RNA sequence inhibits tocopherol biosynthesis.

8. The transgenic plant of claim 7, wherein said heterologous nucleic acid sequences are operably linked to the same promoter.

9. The transgenic plant of claim 7, wherein said heterologous nucleic acid sequences are separated by a loop sequence.

10. The transgenic plant of claim 8, wherein said promoter is a tissue specific promoter.

11. The transgenic plant of claim 8, wherein said promoter is a constitutive promoter.

12. The transgenic plant of claim 7, wherein said heterologous nucleic acid sequences are operably linked to separate promoters.

13. The transgenic plant of claim 7, wherein said transgenic plant comprises at least two heterologous nucleic acid sequences each encoding a double stranded plant RNA sequence, wherein each double stranded plant RNA sequence inhibits the tocopherol biosynthesis of said plant.

14. The transgenic plant of claim 7, wherein one of said heterologous nucleic acid sequences is complementary to an RNA sequence selected from the group consisting of tocopherol cyclase and homogentisate phytyl transferase.

15. A vector comprising heterologous nucleic acid sequences encoding a double stranded plant RNA sequence, wherein said double stranded RNA sequence inhibits tocopherol biosynthesis of a plant.

16. A method for generating biofuels comprising:
a) providing transgenic plant comprising heterologous DNA sequences encoding a double stranded plant RNA to inhibit tocopherol biosynthesis,
b) growing said transgenic plants under such conditions that tocopherol biosynthesis in inhibited and carbon allocation is altered,
c) harvesting said transgenic plant tissues with altered carbon allocation such that a biofuel is generated.

17. The method of claim 16, wherein said transgenic plant is an Arabidopsis species.

18. The method of claim 16, wherein said transgenic plant is a crop plant.

19. The method of claim 16, wherein said transgenic plant comprising heterologous DNA sequences encoding a double stranded plant RNA to inhibit tocopherol biosynthesis inhibits tocopherol cyclase.

20. The method of claim 16, wherein said transgenic plant comprising heterologous DNA sequences encoding a double stranded plant RNA to inhibit tocopherol biosynthesis inhibits homogentisate phytyl transferase.
